(19) United States
(12) Patent Application Publication
(10) Pub. No.: US 2011/0038805 A1
(43) Pub. Date: Feb. 17, 2011

(54) COMPOUNDS COMPRISING PARAMAGNETIC CHELATES ARRANGED AROUND A CENTRAL CORE AND THEIR USE IN MAGNETO RESONANCE IMAGING AND SPECTROSCOPY

(76) Inventors: Andreas Meijer, Oslo (NO); Oskar Axelsson, Lund (SE); Anders Brathe, Sandefjord (NO); Andreas Olsson, Oslo (NO); John Henrik Johansen, Oslo (NO); Duncan George Wynn, Amersham (GB)

Correspondence Address: GE HEALTHCARE, INC., IP DEPARTMENT 101 CARNEGIE CENTER, PRINCETON, NJ, 08540-6231, US

(21) Appl. No.: 12/937,719

(22) PCT Filed: Apr. 17, 2009
(86) PCT No.: PCT/EP09/54579

§ 371 Date: Oct. 14, 2010

(30) Foreign Application Priority Data
Apr. 18, 2008 (EP) 08007584.9

(51) Int. Cl. A61B 5/055; C07D 257/02; A61K 31/555

(52) U.S. Cl. 424/9.363; 540/465; 514/184

(57) ABSTRACT

The present invention relates to novel compounds of formula (I) and (II), compositions comprising compounds of formula (II) and their use as contrast agents in magnetic resonance (MR) imaging (MRI) and MR spectroscopy (MRS).

The present invention relates to novel compounds of formula (I) and (II), compositions comprising compounds of formula (II) and their use as contrast agents in magnetic resonance (MR) imaging (MRI) and MR spectroscopy (MRS).

MR image signal is influenced by a number of parameters that can be divided into two general categories: inherent tissue parameters and user-selectable imaging parameters. Inherent tissue parameters that affect MR signal intensity of a particular tissue are mainly the proton density, i.e. hydrogen nuclei density of that tissue and its inherent T₁ and T₂ relaxation times. Signal intensity is also influenced by other factors such as flow. The contrast between two adjacent tissues, e.g. a tumour and normal tissue depends on the difference in signal between the two tissues. This difference can be maximised by proper use of user-selectable parameters. User-selectable parameters that can affect MR image contrast include choice of pulse sequences, flip angles, echo time, repetition time and use of contrast agents.

Contrast agents work by effecting the T₁, T₂ and/or T₂* relaxation times and thereby influencing the contrast in the images. Information related to perfusion, permeability and cellular density as well as other physiological parameters can be obtained by observing the dynamic behaviour of a contrast agent.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extra cellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

The existent paramagnetic metal chelates that are used as MR contrast agents have a low relaxivity at the 1.5 T magnetic field that is standard in most of today's MR scanners. In 3 T systems which probably will dominate or at least be a substantial fraction of the market in the future, the intrinsic contrast is lower, all T₁ values are higher and the hardware will be faster, so the need for a contrast agent with good performance at 3 T is considerable. In general, the longitudinal relaxivity (r1) of contrast agents falls off at the high magnetic fields of the modern MR scanners, i.e. 1.5 T, 3 T or even higher. This is due to the fast rotational Brownian motion of small molecules in solution which leads to weaker magnetic field coupling of the paramagnetic metal ion to the water molecules than anticipated.

Many attempts have been made to produce contrast agents with high relaxivity by incorporating the paramagnetic metal chelates into larger molecules, such as various polymers. These attempts have been of limited success because of fast internal rotations or segmental motions. Another approach are paramagnetic metal chelates that are bound to or do bind to proteins. However such compounds suffer from pharmacological and pharmacokinetic disadvantages like long excretion time or the risk for interactions with protein bound drugs. Further the leakage through normal endothelium into the interstitium is still substantial.

U.S. Pat. No. 5,820,849 describes chelated complexes attached to globular cascade polymers, however the structures presented therein are not optimized with respect to compactness, rigidity, metal density or a low degree of deformability. It discloses cascade polymer complexes of varying generations, but the structures do not contain any short linker fragments and can not be considered to be rigid as they contain aliphatic linker fragments with very small rotational barriers.

EP 1480979 also discloses complexes attached to globular cascade polymers. The document discloses chelates attached to a core via branching units containing aliphatic segments that obliviate any rigidity imposed to the attached chelates.

The present invention provides novel compounds that perform well as MR contrast agents at high magnetic fields, i.e. above 1.5 T. The novel compounds are dendrimeric rigid structures that have slowly rotating bonds.

Thus, in a first aspect the invention provides compounds of formula (I)

A-(B-L-R-(L-R-(L-R-(L-R-(L′-X)ᵣ)ᵣ)ᵣ)ᵣ)ₙ (I)

wherein
A denotes a core;
B is the same or different and denotes a moiety that constitutes an obstacle for the rotation of the covalent bond between B and L;
L is the same or different and denotes a rigid linker moiety, wherein at least one L is present and under the proviso that L never links directly to another L;
L′ is present or not and if present is the same or different and denotes a linker moiety;
R is the same or different and denotes a branching moiety that reproduces with an individual multiplicity of r, wherein at least one R is present;
X is the same or different and denotes a chelator;
r r is the same or different and denotes the integer 2, 3 or 4; and
n denotes an integer of 3 to 6.

The term “chelator” denotes a chemical entity that binds (complexes) a metal ion to form a chelate. If the metal ion is a paramagnetic metal ion, the chemical entity, i.e. complex formed by said paramagnetic metal ion and said chelator is denoted a “paramagnetic chelate”.

A preferred embodiment of a compound of formula (I) is a compound of formula (II)

A-(B-L-R-(L-R-(L-R-(L-R-(L′-X′)ᵣ)ᵣ)ᵣ)ᵣ)ₙ (II)

wherein
A denotes a core;
B is the same or different and denotes a moiety that constitutes an obstacle for the rotation of the covalent bond between B and L.
L is the same or different and denotes a rigid linker moiety, wherein at least one L is present and under the proviso that L never links directly to another L;
L′ is present or not and if present is the same or different and denotes a linker moiety;
R is the same or different and denotes a branching moiety that reproduces with an individual multiplicity of r, wherein at least one R is present;
X′ is the same or different and denotes a paramagnetic chelate consisting of a chelator X and a paramagnetic metal ion M; and
r r is the same or different and denotes the integer 2, 3 or 4; and
n denotes an integer of 3 to 6.

Hence, the compounds of formula (II) are compounds of formula (I) wherein X is a paramagnetic chelate X′.

In said preferred embodiment, said paramagnetic chelate X′ consists of the chelator X and a paramagnetic metal ion M, said chelator X and paramagnetic metal ion M form a complex which is denoted a paramagnetic chelate.

In the following the term “ . . . X/X′”, e.g. in L′-X/X′ or in the formulae, means that the statement made or the drawn formula is equally suitable for compounds or residues comprising the chelator X or the paramagnetic chelate X′.

The compounds of the present invention according to formulae (I) and (II) are of the dendrimer type. Dendrimers are a class of polymer molecules with a central core with multiple branching arms including branching moieties that reproduces with an individual multiplicity. In the compounds of formulae (I) and (II) each branching arm is terminally linked to a chelator or a paramagnetic chelate. The number of chelators or chelates in the compound depends on the number of branching moieties added to the structure, the multiplicity of the branching moieties and the number of branching arms on the core A. Depending on the number of branching moieties, and assuming that all branching moieties have a multiplicity of two, a compound with four branching arms will comprise of 8, 16, 32 or 64 chelators or chelates. A compound with three branching arms, each with branching moieties with a multiplicity of two, will comprise of 6, 12, 24 or 48 chelators or chelates. A compound of formula (II) with 8 chelates is shown in Figure A. The compound comprises a core A with four branching arms (n is 4), each arm comprising one branching moiety with an individual multiplicity of 2 (r is 2). Hence the compound has the formula A-(B-L-R-(L′-X′)₂)₄. Figure B shows a compound with one additional branching moiety on each of the two arms resulting from the first branching moiety. This compound has 16 chelates and the formula A-(B-L-R-(L-R-(L′-X′)₂)₂)₄.

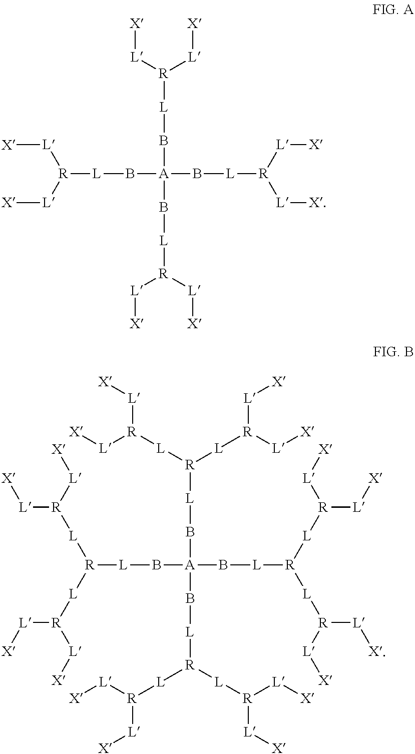

The dendrimer compounds of the present invention show high relaxivity because of the rigidity and compactness of the structure, preventing a fast rotation of the covalent bonds and deformation of the molecule, and allowing a large number of chelates per molecule weight of the molecule.

The core A of the compounds of formula (I) and (II) preferably is a non-polymeric core. In another preferred embodiment, A is a cyclic core or a carbon atom having attached thereto 3 or 6 moieties B, wherein, when 3 moieties B are attached to said carbon atom, the forth valence may be hydrogen or a group selected from amino, hydroxyl, C₁-C₃-alkyl or halogen.

In one embodiment A is preferably a saturated or non-saturated, aromatic or aliphatic ring comprising at least 3 carbon atoms and optionally one or more heteroatoms N, S or O, said ring being optionally substituted with one or more of the following substituents: C₁-C₃-alkyl, optionally substituted with hydroxyl or amino groups, amino or hydroxyl groups or halogen, provided that there are n attachment points left for moieties B.

Preferably, A is an aliphatic saturated or non-saturated 3- to 10-membered ring like cyclopropane, cyclobutane, cycloheptan or cyclohexane, which optionally comprises one or more heteroatoms N, S or O and which is optionally substituted with one or more substituents C₁-C₃-alkyl, optionally substituted with hydroxyl or amino groups, amino or hydroxyl groups or halogen, provided that there are 3 to 6 attachment points left for pendant moieties B. Alternatively, A is an aliphatic 3- to 10-membered ring optionally comprising one or more heteroatoms N, S, or O wherein one or more of the ring carbon atoms are carbonyl groups.

In another preferred embodiment, A is an aromatic single or fused 5-to 10-membered ring optionally comprising one or more heteroatoms N, S or O. Examples for such rings are for instance benzene or naphthalene. The aforementioned rings are optionally substituted with one or more substituents C₁-C₃-alkyl, optionally substituted with hydroxyl or amino groups, amino or hydroxyl groups or halogen, provided that there are at least 3 attachment points left for pendant moieties B.

In another preferred embodiment the core A is an ethyl group. The ethyl group may have attached thereto a maximum of 6 moieties B, wherein, when less than 6 moieties B are attached to said carbon atoms, the remaining valence(s) are hydrogen or a group selected from amino, hydroxyl, C₁-C₃-alkyl or halogen.

Preferred examples of core A are:

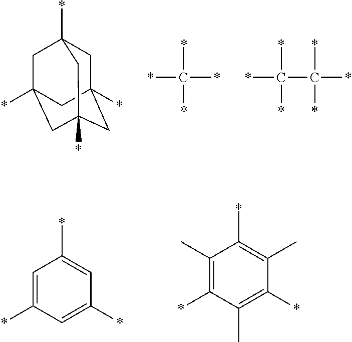

wherein,
* stands for the possible attachment points to B

In the compounds of formulae (I) and (II), B is the same or different and denotes a moiety that constitutes an obstacle for the rotation of the covalent bonds between B and L. This may be achieved by choosing a moiety B whose rotation is hindered by interaction with L, preferably sterical interaction.

Such sterical interaction occurs if B is a bulky moiety like an at least 5-membered carbocyclic or heterocyclic ring or a bicyclic or polycyclic ring. Such sterical interaction may further be promoted by using a bulky moiety B, e.g. the aforementioned bulky moieties which is substituted with C₁-C₃-alkyl, e.g. methyl, ethyl, n-propyl or isopropyl. Such bulky moieties B hinder the rotation of the B moiety due to interaction with L.

In a preferred embodiment B is selected from a residue of an optionally substituted aromatic or non-aromatic 5- to 7-membered carbocyclic or heterocyclic ring like pyridinyl, phenyl, substituted phenyl like benzyl, ethylbenzyl or cyclohexyl. In another preferred embodiment B is selected from a residue of an optionally substituted bicyclical or polycyclic ring like naphthyl or benzimidazolyl. Optional substituents are C₁-C₈-alkyl, hydroxyl, amino or mercapto groups or C₁-C₈-alkyl containing one or more hydroxyl or amino groups like CH₂OH, C₂H₄OH, CH₂NH₂ and/or an oxo-group like CH₂OCH₃ or OC₂H₄OH.

The term “residues of . . . ” in the previous paragraph is chosen since B is attached to A and L. Thus, B is to be seen as a residue.

In a particularly preferred embodiment B is a residue of a 6-membered aromatic ring, preferably a benzene residue.

Preferred examples of B are:

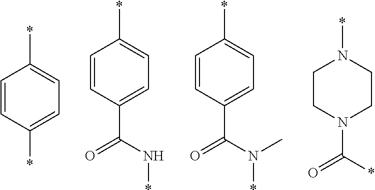

wherein,
* stands for the possible attachment points to A and L

In one embodiment the B moieties can be interconnected by covalent bonds.

Further, compounds of formula (I) and (II) are rigid compounds since the linker moiety L and the branching moieties R exert a rotation restriction.

L denotes a linker moiety that renders the compounds of formulae (I) and (II) compact and rigid. L is a covalent bond or can be chosen from the group:

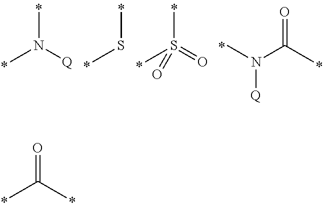

wherein
Q stands for H, C₁-C₈-alkyl, optionally substituted with one or more hydroxyl or amino groups; and
* stands for the possible attachment points to B and R

Preferably L is one of:

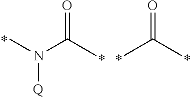

wherein Q and * have the meaning as described above.

Preferably, Q stands for H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

R denotes a branching moiety that reproduces with an individual multiplicity of r wherein r is 2, 3 or 4, with 2 being most preferred. The branching moiety exerts a rotation restriction and hence renders the branching arm rigid. This may be achieved by choosing a moiety R whose rotation is hindered by interaction with L and/or L′, preferably sterical interaction.

Preferred branching moieties are:

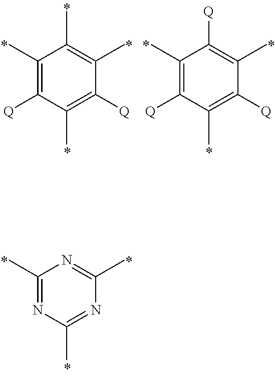

wherein,
Q is the same or different and stands for H, C₁-C₈-alkyl, optionally substituted with one or more hydroxyl or amino groups; and
* stands for the possible attachment points to L, L′ and X/X′.

Preferably all Q are the same and Q is either H or CH₃.

Preferably all R are the same.

In compounds of formula (I) and (II), L′ may be present or not. If L′ is not present, R is directly linked to X (compounds of formula (I)) or X′ (compounds of formula (II)) via a covalent bond. If L′ is present, each L′ is the same or different and denotes a linker moiety, i.e. a moiety that is able to link R and X/X′.

Preferably L′ is selected from:

Linker moieties *—(CZ¹Z²)ₘ—*

Wherein
* stands for the possible attachment points to X/X′ and R; and
m is an integer of 1 to 6; and
Z¹ and Z² independently of each other denote a hydrogen atom, a hydroxyl group or a C₁-C₈-alkyl group optionally substituted by hydroxyl, amino or mercapto groups, e.g. CH₂OH and CH₂CH₂NH₂ and/or optionally comprising an oxo-group, e.g. CH₂OCH3 and OCH₂CH₂OH.

Linker moieties *—CZ¹Z²—CO—N(Q)-* which are more preferred linker moieties,

wherein
* stands for the possible attachment points to X/X′ and R; and
Z¹, Z² have the meaning mentioned above
Q stands for H, C₁-C₈-alkyl, optionally substituted with one or more hydroxyl or amino groups.

In a preferred embodiment, Z¹ and Z² are hydrogen or Z¹ is hydrogen and Z² is methyl and Q is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

Linker moieties *—CO—N(Q)-*

wherein
* stands for the possible attachment points to X/X′ and R; and
Q has the meaning mentioned above.

Linker moieties *—CO—CZ¹Z²—N(Q)-*

wherein
* stands for the possible attachment points to X/X′ and R; and
Z¹, Z² and Q have the meaning mentioned above

In a preferred embodiment, Z¹ and Z² are hydrogen or Z¹ is hydrogen and Z² is methyl and Q is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

Linker moieties which are amino acid residues *—CH₂—CO—NH—CH(Z³)CO—NH—*

wherein
* stands for the possible attachment points to X/X′ and R; and
Z³ stands for the side group of the naturally occurring α-amino acids.

Further preferred examples of L′ are or comprise residues of benzene or N-heterocycles such as imidazoles, triazoles, pyrazinones, pyrimidines and piperidines.

Preferably, if present, all L′ are the same.

Most preferably L′ is selected from:

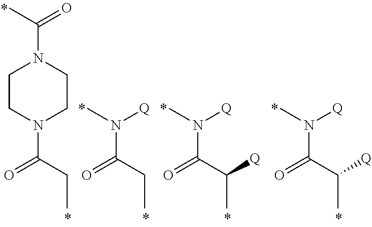

wherein,
Q is the same or different and stands for H, C₁-C₈-alkyl, optionally substituted with one or more hydroxyl or amino groups; and
* stands for the possible attachment points to X/X′ and R

In compounds of formula (I), X is the same or different and denotes a chelator. In the preferred embodiment of compounds of formula (II), X is X′ which stands for a paramagnetic chelate, i.e. a chelator X which forms a complex with a paramagnetic metal ion M. Numerous chelators X which form complexes with paramagnetic metal ions M are known in the art. Preferably, X is a cyclic chelator of formula (III):

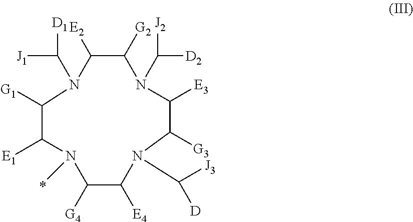

wherein
* denotes the attachment of L′, if present, or the core, if L′ is not present;
E₁ to E₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃ or C(O)N(CH₂CH₃)₂;
G₁ to G₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃, or C(O)N(CH₂CH₃)₂;
D₁ to D₃ independent of each other is selected from H, OH, CH₃, CH₂CH₃, CH₂OH, CH₂OCH₃, OCH₂CH₃, OCH₂CH₂OH or OCH₂C₆H₅; and
J₁ to J₃ independent of each other is selected from COOH, P(O)(OH)₂, P(O)(OH)CH₃, P(O)(OH)CH₂CH₃, P(O)(OH)(CH₂)₃CH₃, P(O)(OH)Ph, P(O)(OH)CH₂Ph, P(O)(OH)OCH₂CH₃, CH(OH)CH₃, CH(OH)CH₂OH, C(O)NH₂, C(O)NHCH₃, C(O)NH(CH₂)₂CH₃, OH or H.

Preferred chelators X are residues of diethylenetriaminopentaacetic acid (DTPA), N-[2-[bis(carboxymethyl)amino]-3-(4-ethoxyphenyl)propyl]-N-[2-[bis(carboxymethyl)-amino]ethyl]-L-glycine (EOB-DTPA), N,N-bis[2-[bis(carboxymethyl)amino]-ethyl]-L-glutamic acid (DTPA-Glu), N,N-bis[2-[bis(carboxymethyl)amino]-ethyl]-L-lysine (DTPA-Lys), mono- or bis-amide derivatives of DTPA such as N,N-bis[2-[carboxymethyl[(methylcarbamoyl)methyl]amino]-ethyl]glycine (DTPA-BMA), 4-carboxy-5,8,11-tris(carboxymethyl)-1-phenyl-2oxa-5,8,11-triazatridecan-13-oic acid (BOPTA), DTPA BOPTA, 1,4,7,10-tetraazacyclododecan-1,4,7-triactetic acid (DO3A), 1,4,7,10-tetraazacyclododecan-1,4,7,10-tetraactetic acid (DOTA), ethylenediaminotetraacetic acid (EDTA), 10-(2-hydroxypropyl)-1,4,7,10-tetraazacyclododecan-1,4,7-triacetic acid (HPDO3A), 2-methyl-1,4,7,10-tetraazacyclododecan-1,4,7,10-tetraacetic acid (MCTA), tetramethyl-1,4,7,10-tetraazacyclododecan-1,4,7,10-tetraacetic acid (DOTMA), 3,6,9,15-tetraazabicyclo[9.3.1]pentadeca-1(15), 11,13-triene-3,6,9-triacetic acid (PCTA), PCTA12, cyclo-PCTA12, N,N′Bis(2-aminoethyl)-1,2-ethanediamine (TETA), 1,4,7,10-tetraazacyclotridecane-N,N′,N″,N′″-tetraacetic acid (TRITA), 1,12-dicarbonyl, 15-(4-isothiocyanatobenzyl) 1,4,7,10,13-pentaazacyclohexadecane-N,N′,N″ triaceticacid (HETA), 1,4,7,10-tetraazacyclododecane-N,N′,N″,N′″-tetraacetic acid mono-(N-hydroxysuccinimidyl)ester (DOTA-NHS), N,N′-Bis(2-aminoethyl)-1,2-ethanediamine-N-hydroxy-succinimide ester (TETA-NHS), [(2S,5S,8S,11S)-4,7,10-tris-carboxymethyl-2,5,8,11-tetramethyl-1,4,7,10-tetraazacyclododecan-1-yl]acetic acid (M4DOTA), [(2S,5S,8S,11S)-4,7-bis-carboxymethyl-2,5,8,11-tetramethyl-1,4,7,10-tetraazacyclo-dodecan-1-yl]acetic acid, (M4DO3A), (R)-2-[(2S,5S,8S,11S)-4,7,10-tris-((R)-1-carboxyethyl)-2,5,8,11-tetramethyl-1,4,7,10-tetraazacyclododecan-1-yl]propionic acid (M4DOTMA), 1 O-Phosphonomethyl-1,4,7,1-O-tetraazacyclododecane-1,4,7-triacetic acid (MPDO3A), hydroxybenzyl-ethylenediamine-diacetic acid (HBED) and N,N′-ethylenebis-[2-(o-hydroxyphenolic)glycine] (EHPG).

The term “residues of . . . ” in the previous paragraph is chosen since the chelator is attached to the remainder of the molecule that represents compounds of formula (I) and (II), thus X is to be seen as a residue. The attachment point of X to said remainder of the molecule that represents compounds of formula (I) and (II) may be any suitable point, e.g. a functional group like a COOH group in a chelator like DTPA, EDTA or DOTA or an amino group in a chelators like DTPA-Lys, but also a non-functional group like a methylene group in a chelators like DOTA.

Suitable chelators X and their synthesis are described in e.g. EP-A-071564, EP-A-448191, WO-A-02/48119, U.S. Pat. No. 6,399,043, WO-A-01/51095, EP-A-203962, EP-A-292689, EP-A-425571, EP-A-230893, EP-A-405704, EP-A-290047, U.S. Pat. No. 6,123,920, US-A-2002/0090342, U.S. Pat. No. 6,403,055, WO-A-02/40060, U.S. Pat. No. 6,458,337, U.S. Pat. No. 6,264,914, U.S. Pat. No. 6,221,334, WO-A-95/31444, U.S. Pat. No. 5,573,752, U.S. Pat. No. 5,358,704 and US-A-2002/0127181, the content of which are incorporated herein by reference.

In a more preferred embodiment of the present invention X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

In a particularly preferred embodiment X is selected from residues of DTPA, DOTA, BOPTA, DO3A, HPDO3A, DOTMA, PCTA, DTPA BMA, M4DOTA or M4DO3A.

As stated above, in a preferred embodiment of X, i.e. X′, the chelator X forms a complex, i.e. paramagnetic chelate, with a paramagnetic metal ion M. Suitably, M is a paramagnetic ion of a transition metal or a lanthanide metal, i.e. metals of atomic numbers 21 to 29, 42, 43, 44 or 57 to 71. More preferred, M is a paramagnetic ion of Mn, Fe, Co, Ni, Eu, Gd, Dy, Tm and Yb, particularly preferred a paramagnetic ion of Mn, Fe, Eu, Gd and Dy. Most preferably M is selected from Gd³⁺, Mn²⁺, Fe³⁺, Dy³⁺ and Eu³⁺ with Gd³⁺ being the most preferred paramagnetic ion M.

Preferably all B are the same and/or all L are the same and/or all R are the same and/or all L′ are the same and/or all X/X′ are the same.

Preferred examples of compounds of formula (II) are:

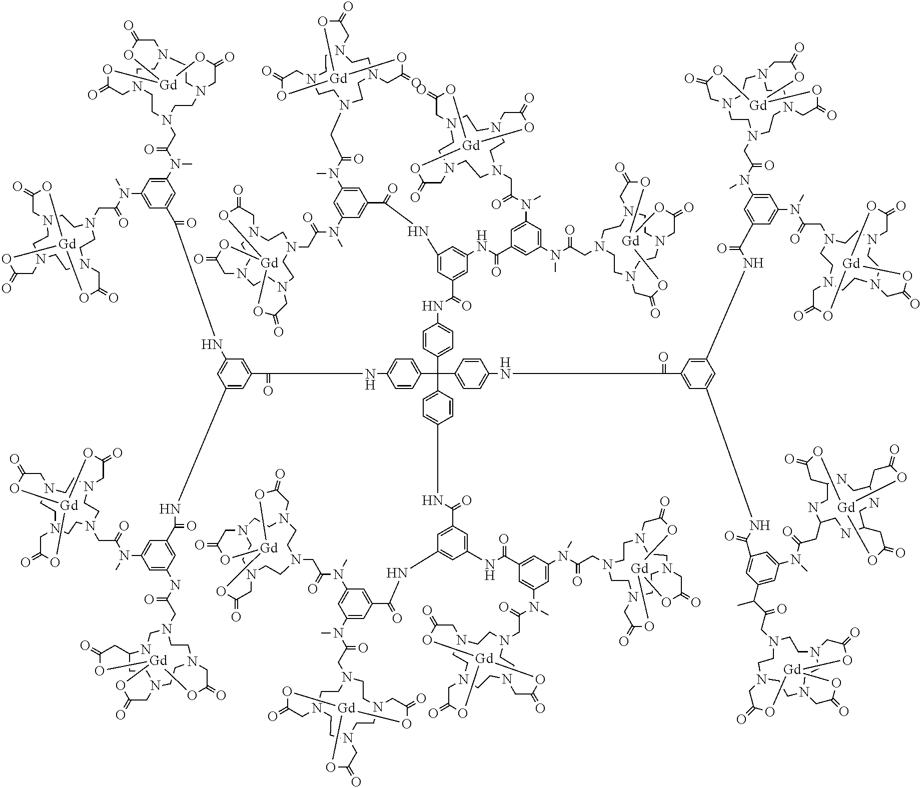

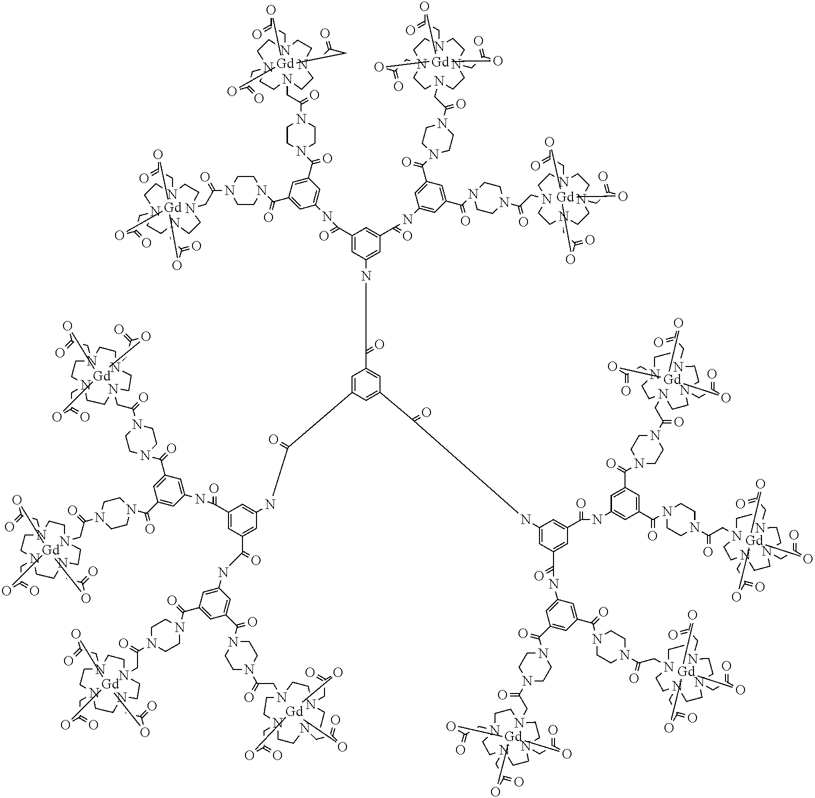

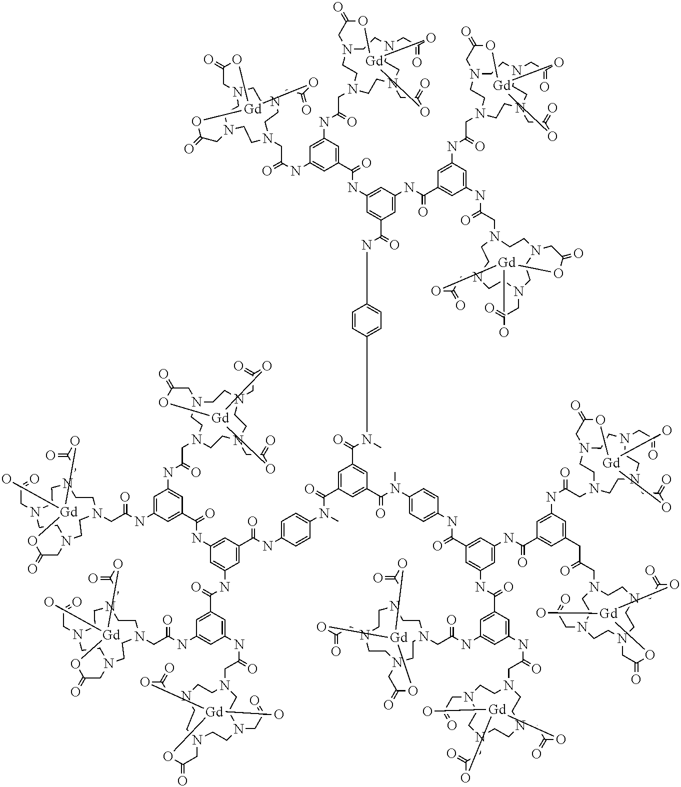

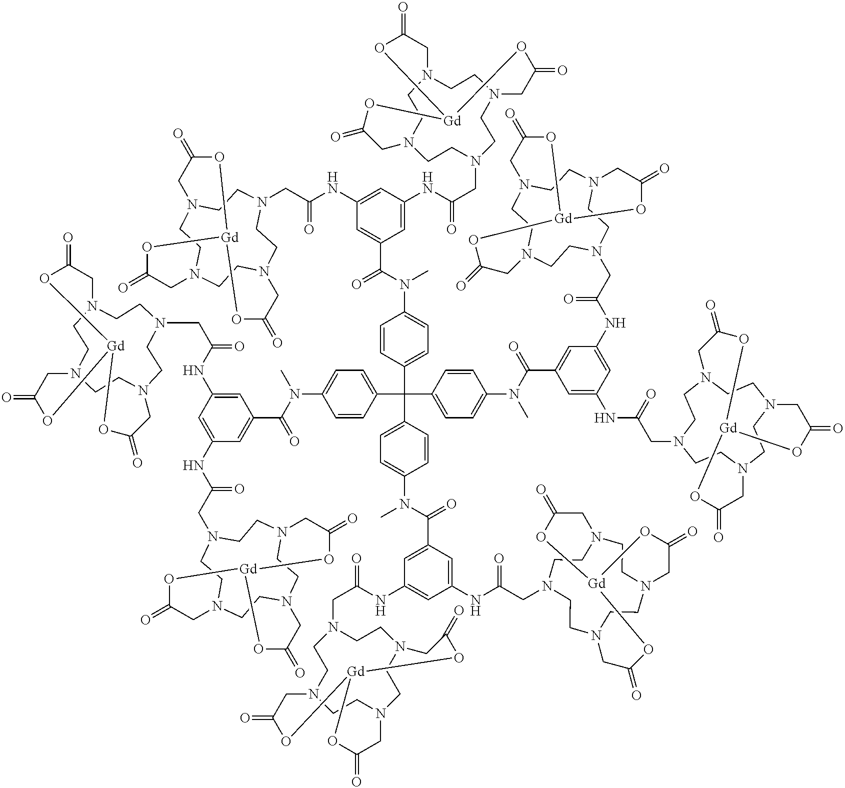

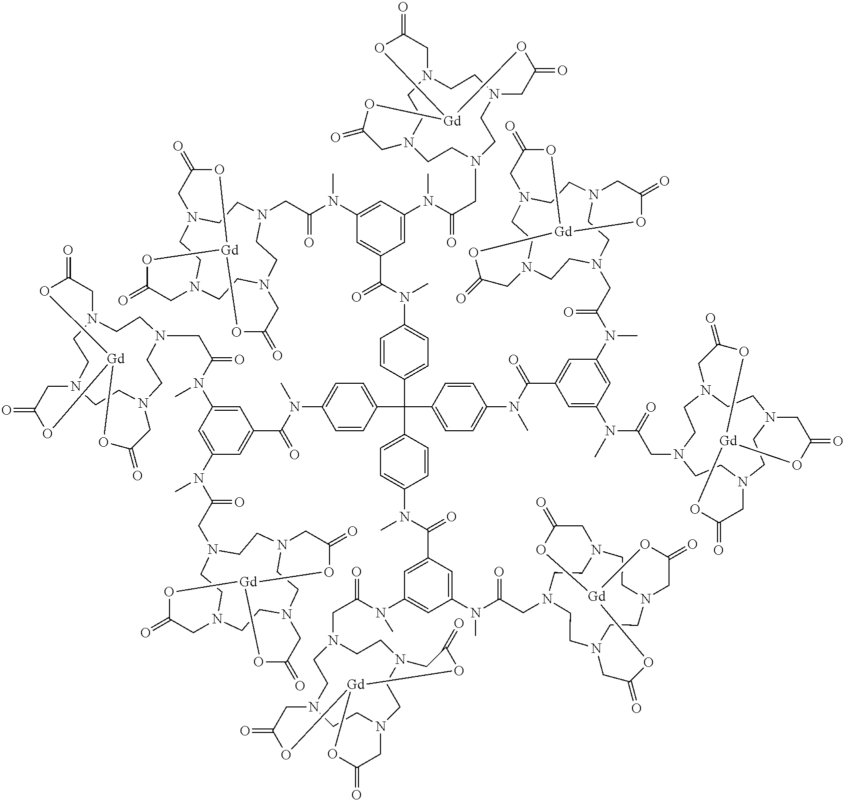

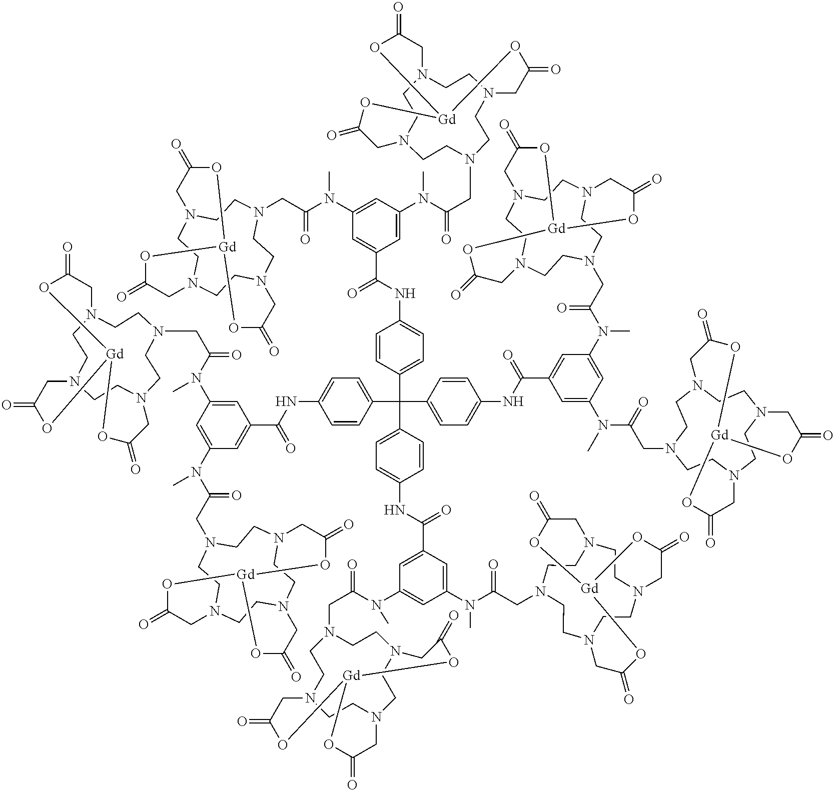

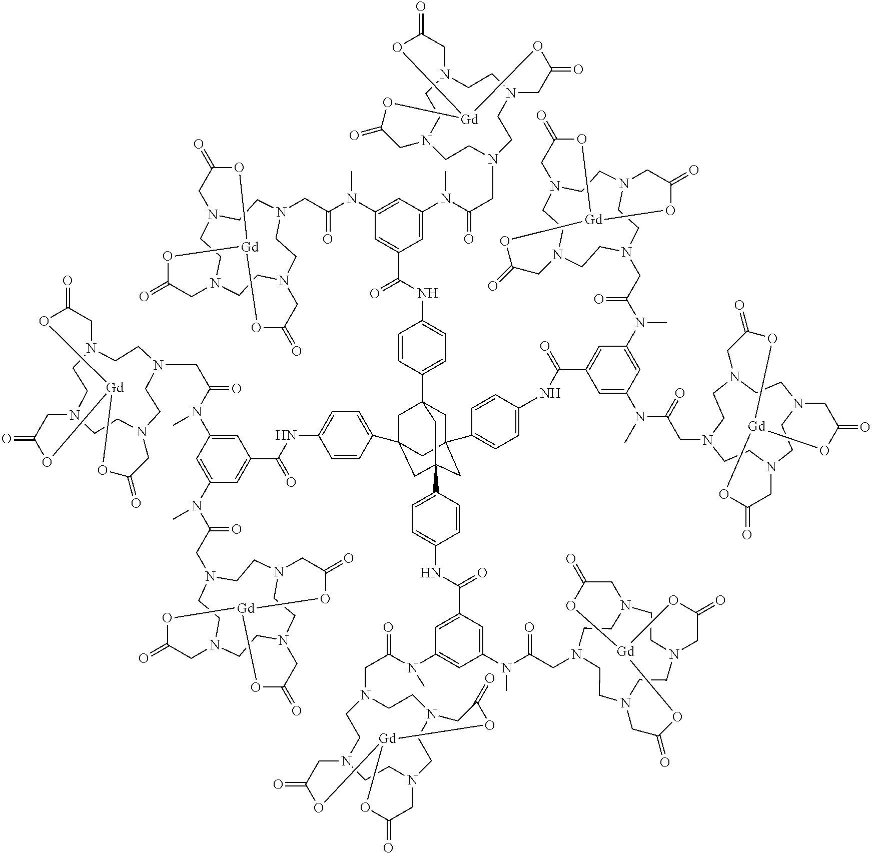

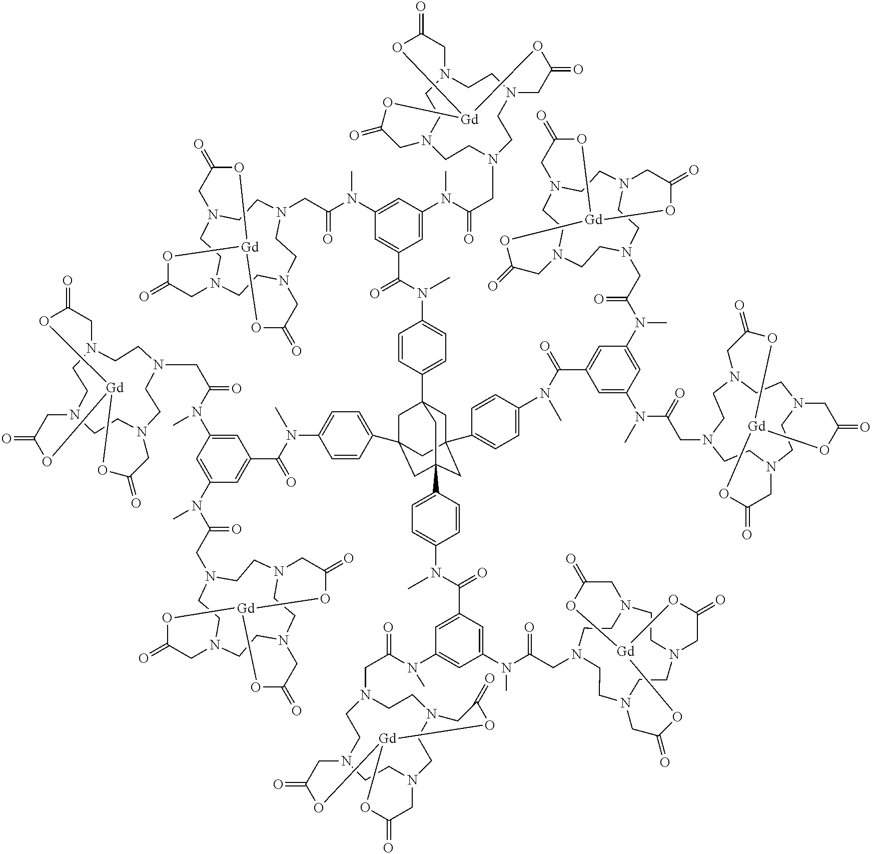

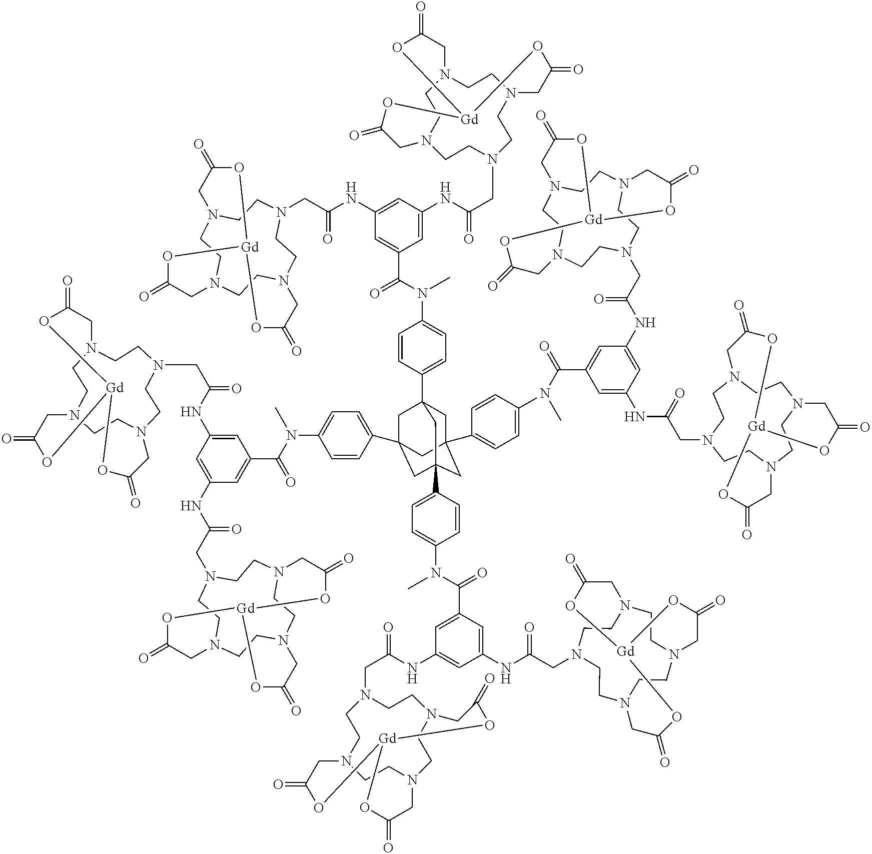

The compounds of formula (I) and (II) can be synthesized by several synthetic pathways known to the skilled artisan from commercially available starting materials by the following generalized process.

The compounds are preferably synthesized by a convergent approach where the individual building blocks are combined and attached to the core structure. In synthesizing a compound of formula A-(B-L-R-(L-R-(L-R-(L-R-(L′-X)ᵣ)ᵣ)ᵣ)ᵣ)ₙ, for example a precursor to the core A can be attached to a precursor to the moiety B. The attachment process is preferably based on an amide bond approach where one of the building blocks is equipped with an amine group and the other building block is equipped with an activated carboxylic acid. By reacting the two building blocks an amide bond will be formed. Alternatively an A-(B)ₙ block that is commercially available is provided.

The attached B moiety is also equipped with additional reactive groups albeit in a protected form. Examples of such are azide-, nitro-, amide- and carbamate-groups, which can be transformed in to an amine group, and ester-groups which can be transformed into an activated carboxylic acid group. The formed A-(B)ₙ building block can then be transformed into an activated form by modification of the latent protective groups, on the B moieties, into functional groups suitable for further attachment.

The A-(B)ₙ block can then be attached to a R-(L′-X)ᵣ block in a convergent fashion, by forming an amide bond using the same methodology as when attaching the A and B building blocks. The R-(L′-X)r block is preferably produced from a R moiety by sequential attachment of a precursor to L′ followed by X. The precursor of L′ is preferably equipped with a leaving group that can be displaced by a nucleophilic X moiety. Examples of leaving groups are: chloride-, bromide-, tosyl-, mesyl- and triflate-groups. The attachment process is well known for the one skilled in the art and can be described as a nucleophilic substitution reaction.

The R moiety of the R-(L′-X)r block is then preferably modified to be attached to the A-(B)ₙ block by an amide bond approach. The purpose of the modification is to prepare the R moiety, of the R-(L′-X)ᵣ block, to be attached to the B moiety of the A-(B)n block. The modification is analogous to the one previously described for the attachment of moiety B to core A, and will either form an activated carboxylic acid or an amine functional group.

By combining the A-(B)ₙ and R-(L′-X)ᵣ blocks, A-(B-L-R-(U-X)ᵣ)ₙ is formed.

If a compound with more branching moieties R is produced then two R moieties, suitable prepared, are combined by forming an amide bond. The preparation involves transformation of protected functional groups into an amine group and an activated carboxylic acid, as previously described. The formed R-(L-R)ᵣ block is then sequentially reacted with a precursor to L′ and X, as previously described to give a R(-L-R-(L′-X)ᵣ)ᵣ block. Alternatively the R-(L-R-(L′-X)ᵣ)ᵣ block can be produced by combining a suitable activated R and R-(L′-X)ᵣ blocks using the amide bond methodology described above.

The R-(L-R-(L′-X)ᵣ)ᵣ block is then prepared for attachment to block A-(B)ₙ, as previously described, to give A-(B-L-R-(L-R-(L′-X)ᵣ)ᵣ)ₙ by the formation of an amide bond.

To give a compounds of general formula A-(B-L-R-(L-R-(L-R-(L-R-(L′-X′)ᵣ)ᵣ)ᵣ)ᵣ)ₙ, the chelator X can be transformed into a chelate X′ by complexation with a metal ion at any time of the synthesis.

Preferably the chelator X is transformed into a chelate X′ after the synthesis of the compound of formula A-(B-L-R-(L-R-(L-R-(L-R-(L′-X)ᵣ)ᵣ)ᵣ)ᵣ)ₙ.

The compounds of formula (II) and preferred embodiments thereof may be used as MR contrast agents. For this purpose, the compounds of formula (II) are formulated with conventional physiologically tolerable carriers like aqueous carriers, e.g. water and buffer solution and optionally excipients.

Hence in a further aspect the present invention provides a composition comprising a compound of formula (II) or preferred embodiments thereof and at least one physiologically tolerable carrier.

In a further aspect the invention provides a composition comprising a compound of formula (II) and preferred embodiments thereof and at least one physiologically tolerable carrier for use as MR imaging agent or MR spectroscopy agent.

To be used as agents for MR imaging or spectroscopy of the human or non-human animal body, said compositions need to be suitable for administration to said body. Suitably, the compounds of formula (II) or preferred embodiments thereof and optionally pharmaceutically acceptable excipients and additives may be suspended or dissolved in at least one physiologically tolerable carrier, e.g. water or buffer solutions. Suitable additives include for example physiologically compatible buffers like tromethamine hydrochloride, chelators such as DTPA, DTPA-BMA or compounds of formula (I) or preferred embodiments thereof, weak complexes of physiologically tolerable ions such as calcium chelates, e.g. calcium DTPA, CaNaDTPA-BMA, compounds of formula (I) or preferred embodiments thereof wherein X forms a complex with Ca²⁺ or CaNa salts of compounds of formula (I) or preferred embodiments thereof, calcium or sodium salts like calcium chloride, calcium ascorbate, calcium gluconate or calcium lactate. Excipients and additives are further described in e.g. WO-A-90/03804, EP-A-463644, EP-A-258616 and U.S. Pat. No. 5,876,695, the content of which are incorporated herein by reference.

Another aspect of the invention is the use of a composition comprising a compound of formula (II) or preferred embodiments thereof and at least one physiologically tolerable carrier as MR imaging agent or MR spectroscopy agent.

Yet another aspect of the invention is a method of MR imaging and/or MR spectroscopy wherein a composition comprising a compound of formula (II) or preferred embodiments thereof and at least one physiologically tolerable carrier is administered to a subject and the subject is subjected to an MR procedure wherein MR signals are detected from the subject or parts of the subject into which the composition distributes and optionally MR images and/or MR spectra are generated from the detected signals.

In a preferred embodiment, the subject is a living human or non-human animal body.

In a further preferred embodiment, the composition is administered in an amount which is contrast-enhancing effective, i.e. an amount which is suitable to enhance the contrast in the MR procedure.

In a preferred embodiment, the subject is a living human being or living non-human animal being and the method of MR imaging and/or MR spectroscopy is a method of MR tumour detection or a method of tumour delineation imaging.

In another embodiment, the subject is a living human or non-human animal being and the method of MR imaging and/or MR spectroscopy is a method of MR angiography, more preferred a method of MR peripheral angiography, renal angiography, supra aortic angiography, intercranial angiography or pulmonary angiography.

In another aspect, the invention provides a method of MR imaging and/or MR spectroscopy wherein a subject which had been previously administered with a composition comprising a compound of formula (II) or preferred embodiments thereof and at least one physiologically tolerable carrier is subjected to an MR procedure wherein MR signals are detected from the subject or parts of the subject into which the composition distributes and optionally MR images and/or MR spectra are generated from the detected signals.

The term “previously been administered” means that the method as described above does not contain an administration step of said composition to said subject. The administration of the composition has been carried out previous to the method as described above, i.e. before the method of MR imaging and/or MR spectroscopy according to the invention is commenced.

The invention is illustrated by the following examples.

### EXAMPLES

### Example 1

### Synthesis of a Compound According to the Present Invention with Formula A-(B-L-R-(L-R-(L′-X′)₂)₂)₄

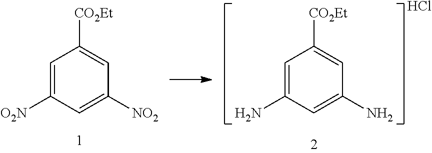

A Buchi reactor was charged with compound 1 (20 g, 83 mmol, CAS: 618-71-3) dissolved in ethanol (500 mL), water (250 mL) and fuming HCl (5.1 mL, 37%). Pd/C (1 g, 10%) was added and the reactor was charged with hydrogen gas at 9 bar. After 20 h of vigorous stirring the reaction mixture was filtered and concentrated. The residue was dissolved in methanol (30 mL) and diethyl ether was added until precipitation occurred. The precipitate was filtered off and dried under vaccum to give 8.44 g (40%) of compound 2 as a fine powder. The structure was confirmed by NMR.

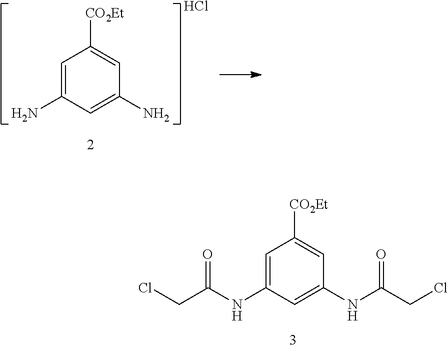

To compound 2 (7.7 g, 35.5 mmol) was added acetonitrile (70 mL, dried Ms4 Å). Chloroacetylchloride (8.5 mL, 0.11 mol) was added drop wise to vigorously stirred slurry under nitrogen atmosphere. After 20 h additional acetonitrile (70 mL, dried Ms4 Å) was added to reaction mixture and the white slurry was added slowly to a vigorously stirred buffer (1 L, 0.3 M, KH₂PO₄, pH adjusted to 7) containing 2 L of ice. The resulting fine white precipitate was filtered off and rinsed with water. The precipitate was dried under vacuum at 40° C. to give 10.8 g (91%) of compound 3. The structure was confirmed by NMR.

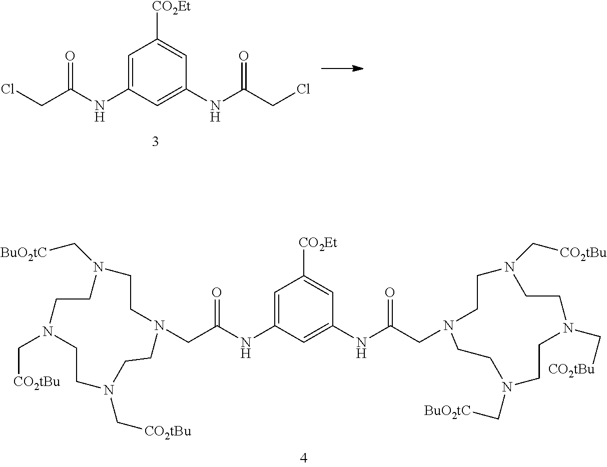

To the HBr salt of DO3A (51.1 g, 86 mmol, for example synthesised as described in WO 2006112723 A1) was added dichloromethane (150 mL, Dried Ms4 Å) followed by N,N,N,N-tetramethylguanidine (10.8 mL, 86 mmol). After two hours the reaction mixture was concentrated and diethyl ether (400 mL) was added to form a white fine slurry. The precipitate was filtered off and the filtrate was concentrated to give a yellow oil (37 g). The oil was then dissolved in acetonitrile (50 mL, dried Ms4 Å) and compound 3 (10.9 g, 32.8 mmol) was added followed by diisopropylethylamine (11.4 mL, 66 mmol). The reaction mixture was refluxed under nitrogen atmosphere for 24 h and then concentrated. The reaction mixture was then extracted using ethyl acetate (350 mL) and a buffer (350 mL, 15 mM, NaH₂PO₄, pH adjusted to 7.5). The aqueous phase was extracted again with ethyl acetate (350 mL). The combined organic phases were dried using Na₂SO₄ and then filtered and concentrated to give crude 4 (43.2 g). The structure was confirmed by LC-MS.

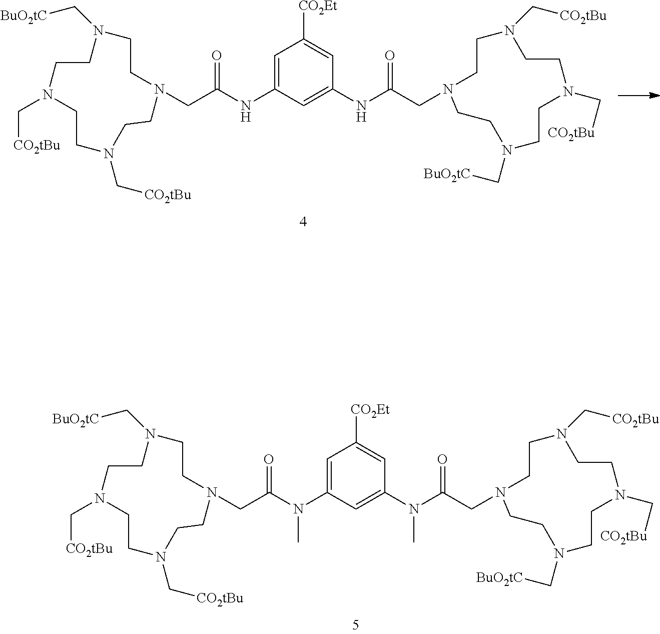

Compound 4 (43 g, 33.3 mmol) was dissolved in THF (400 mL, dried Al₂O₃) and then iodomethane (20.9 mL, 0.33 mol) was added under nitrogen atmosphere. NaH (5.3 g, 60%, 0.2 mol, washed repeatedly with heptane and added as a slurry in 50 mL heptane) was then added portion wise (5 mL) during 20 minutes. After 3.5 h the reaction mixture was concentrated to give crude 5 which were used directly in the next step. The structure was confirmed by LC-MS.

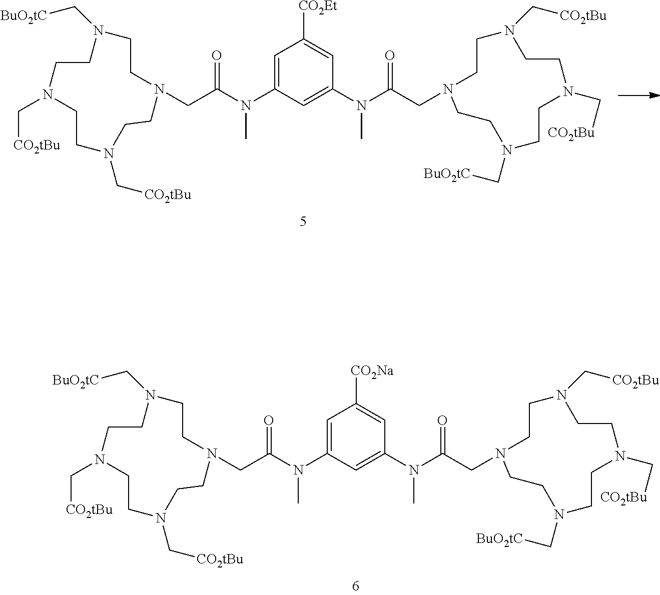

Compound 5 (2.1 g 1.6 mmol) was dissolved in THF (10 mL, Filtered Al₂O₃) and added to water (10 mL) to form an oily suspension. pH was adjusted continuously with NaOH (1M) never exceeding 13. After 24 h the pH of reaction mixture was adjusted to 11 and THF was slowly evaporated off using a rotavapor at 30° C. Eventually a precipitate was formed and filtered off to give 1.05 g (49%) of a fine white powder. The precipitate contained water and was dried by dissolving in THF (Filtered Al₂O₃) and stirring with MS4 Å (3.5 g) for 24 h. After filtration and concentration 1 g (49%) of a fine white powder was obtained. IR indicated no water, and the structure was confirmed by LC-MS.

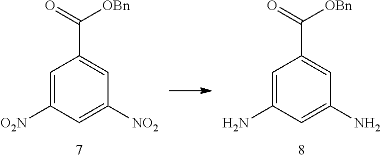

Iron powder (61.2 g; 325 mesh), water (61 g) and acetic acid (0.9 g) was weighed into a flask, and toluene (43 ml) was added. The mixture was stirred mechanically, and heated to reflux. 3,5-dinitrobenzylbenzoate (27.65 g, 91 mmol, CAS: 10478-07-6) dissolved in toluene (43 ml), by heating, was added to the iron slurry (exothermic reaction, caution). After 2 hours, the reaction mixture was cooled to room temperature. More toluene was added, and the mixture was filtered boiling hot. The dissolved product rapidly solidified in the receiving flask. The solid was filtered off, the toluene solution was dried with MgSO4 and evaporated on rotavapor. The material was recrystallized from benzene (40 ml) and hexane (15 ml). to give 12.5 g (56%) of compound 8.

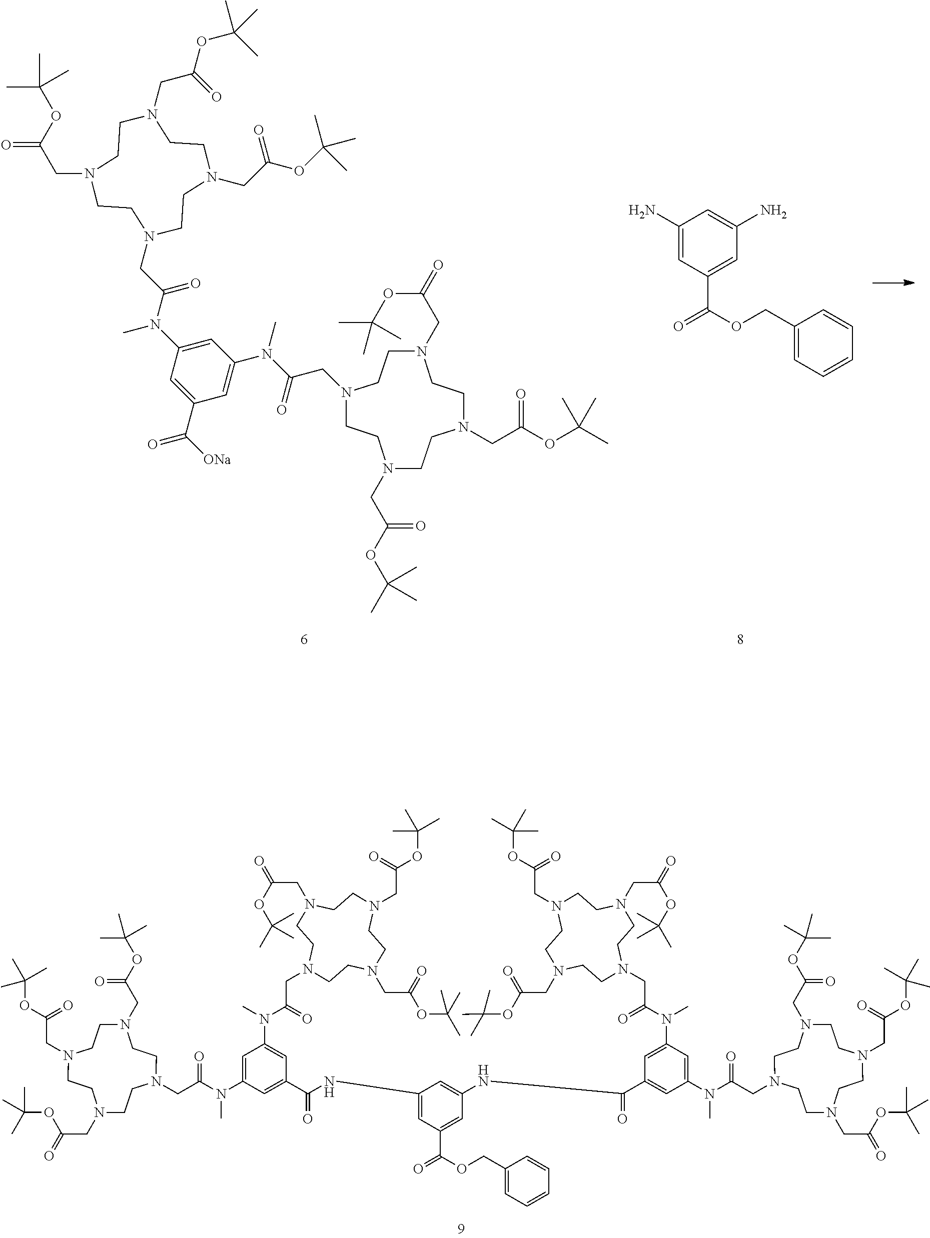

To a solution of SOCl₂ (1.7 mL, 22.9 mmol) in dichloromethane (100 mL) was added drop wise a solution of compound 6 (8.3 g, 6.3 mmol) in dichloromethane (20 mL) under argon atmosphere. After 1 h the reaction mixture was concentrated and the formed oil was azeotroped three times with acetonitrile (3*100 mL) to give an amorphous solid. To the crude reaction mixture was added acetonitrile (100 mL) under argon atmosphere. Compound 8 (640 mg, 2.6 mmol) was added to the stirred slurry. After 1 h the reaction mixture was concentrated to give crude compound 9 (9 g). The structure was confirmed by LC-MS.

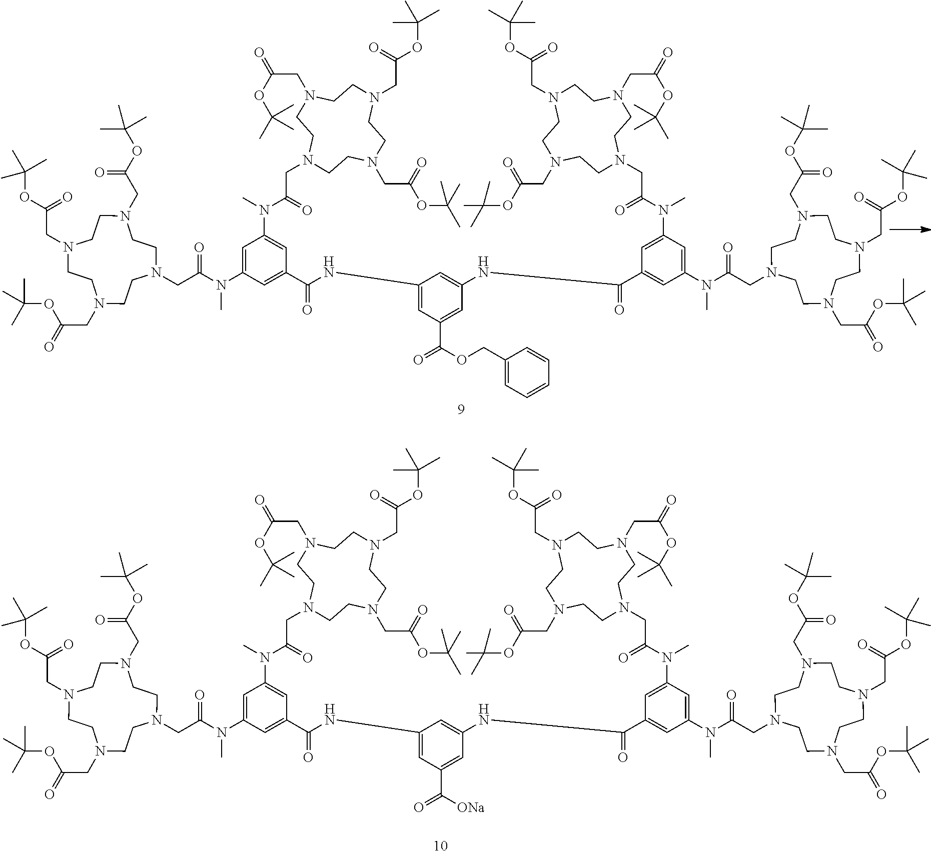

To compound 9 (4.9 g, 1.76 mmol) was added a water:THF:acetonitrile mixture (30 mL:30 mL:30 mL), the pH of the suspension was continuously adjusted with NaOH (1M) never exceeding 13. After 48 h the pH of the reaction mixture was adjusted to 6 (4M HCl) and the organic solvents were partially removed (mainly THF) on a rotavapor (at 40° C.) until an oil crashed out. The aqueous phase was decanted and the remaining oil was purified on preparative HPLC. The pH of the pure fractions was adjusted to 10 and the combined solutions were evaporated until an oil crashed out. The oil solidified upon mechanical grinding with spatula and the formed precipitate was dried under vacuum to give compound 10 as a fine white powder (3.5 g). The structure was confirmed by LC-MS.

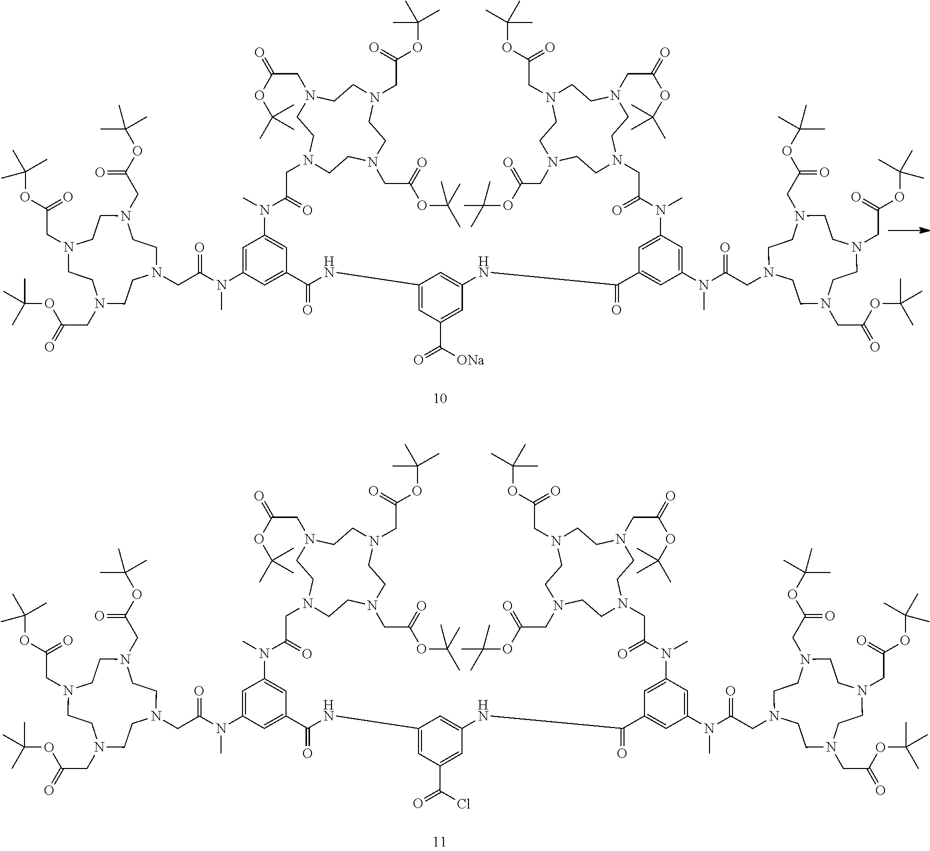

To a solution of SOCl₂ (215 uL, 2.94 mmol) in dichloromethane (10 mL) was added drop wise a solution of compound 10 (1000 mg, 0.368 mmol) in dichloromethane (9 mL). After 30 min the reaction mixture was cooled to 0° C. and then extracted with an ice cold saturated aqueous NaCl (3*30 mL) solution. The resulting emulsion was centrifuged and the organic phase was decanted and dried using MgSO₄ (2 g). The organic phase was filtered and concentrated to give crude acid chloride 11 which was used immediately in the next step. The structure was confirmed by LC-MS.

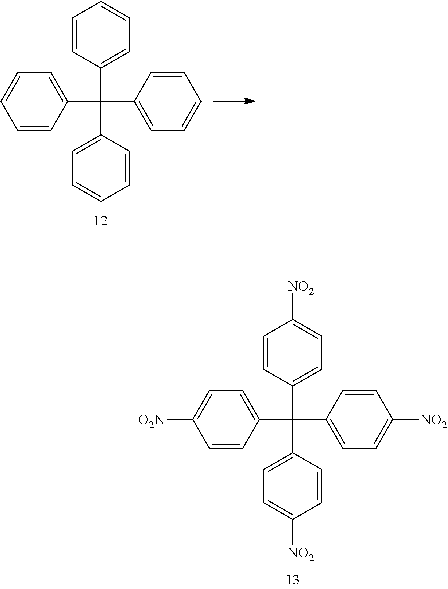

Fuming nitric acid (54 ml) was cooled to −10° C. Tetraphenylmethane 12 (10 g, 31 mmol, CAS: 630-7-6-2) was added slowly over 17 minutes. Acetic anhydride (17 ml) was then added over 7 minutes followed by acetic acid (35 ml). After stirring on ice for 45 minutes, more acetic acid was added (70 ml), and precipitated material was filtered off on a glass sinter and washed with acetic acid. The collected solid (light yellow) was partially recrystallized from THF (30 ml) and air dried overnight to give compound 13 11 g (71%) as a cream coloured solid. The structure was confirmed by NMR.

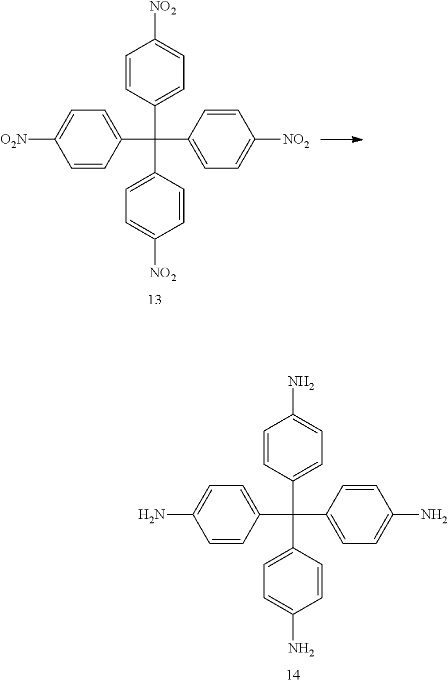

Compound 13 (8.8 g, 17.6 mmol) and Pd on C (0.8 g, 10%) were suspended in THF (500 ml) in a Buchi hydrogenation apparatus. The apparatus was sealed, and 10 bar H₂ was applied at ambient temperature. Reaction continued overnight under vigorous stirring. The reaction mixture was filtered through a celite pad. The pale yellow solution was evaporated on rotavapor to give compound 14 6.3 g (94%) as a beige powder. The structure was confirmed by NMR.

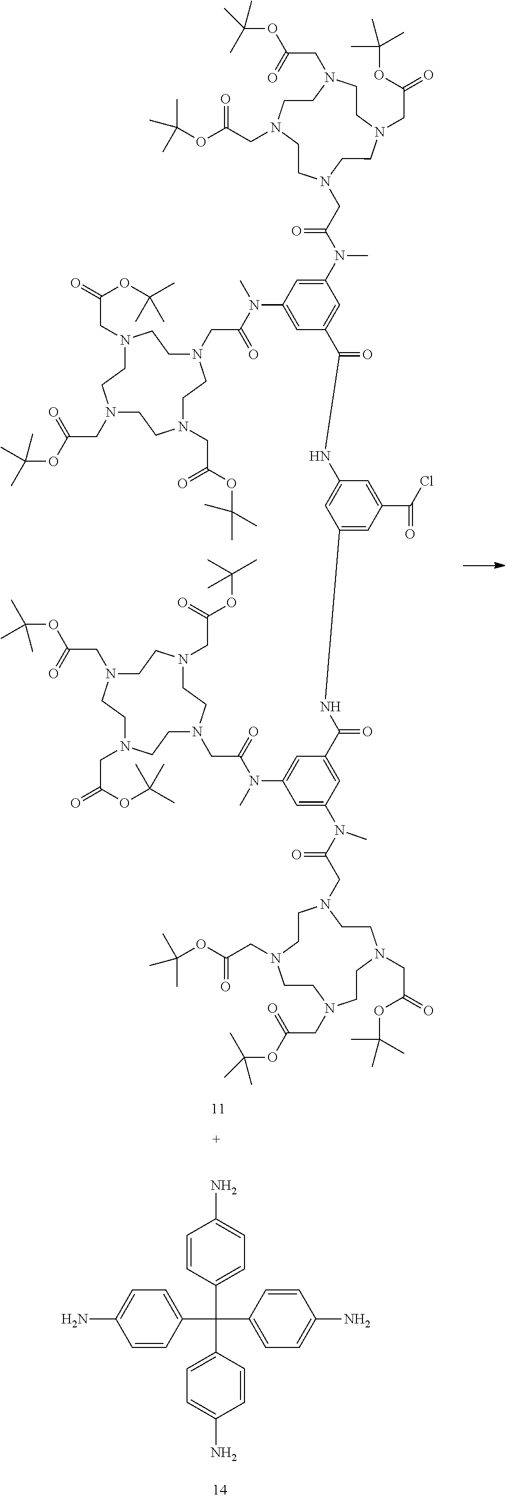

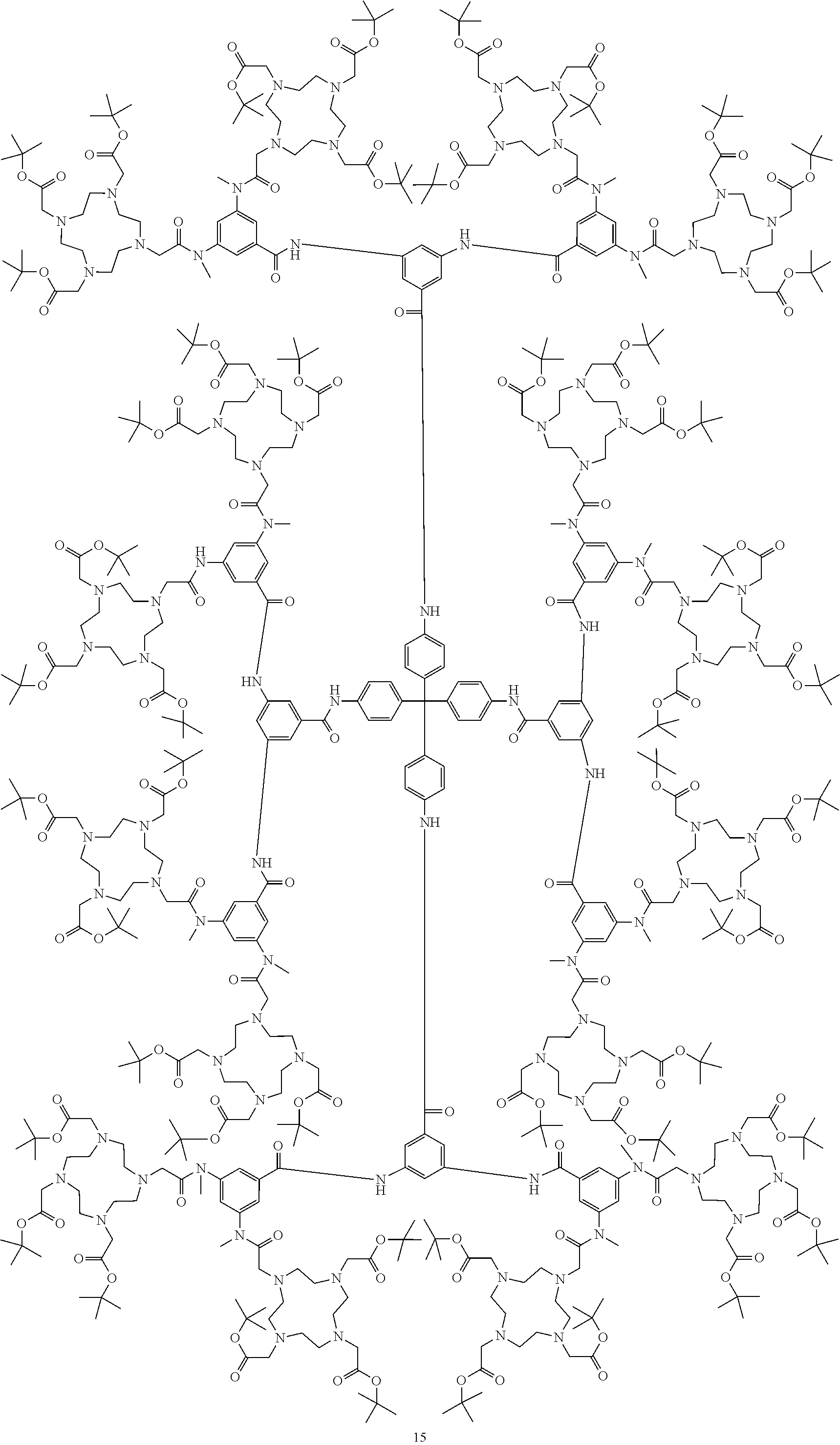

To crude 11 (1000 mg, 0.368 mmol) in dichloromethane (15 mL) and MS4 Å (0.5 g) was added compound 14 (22.2 mg, 58 umol) followed by diisopropylethylamine (360 uL, 2.1 mmol) under argon atmosphere and vigorous stirring. After 20 h the reaction mixture was filtered and concentrated to give crude compound 15. The structure was confirmed by LC-TOF.

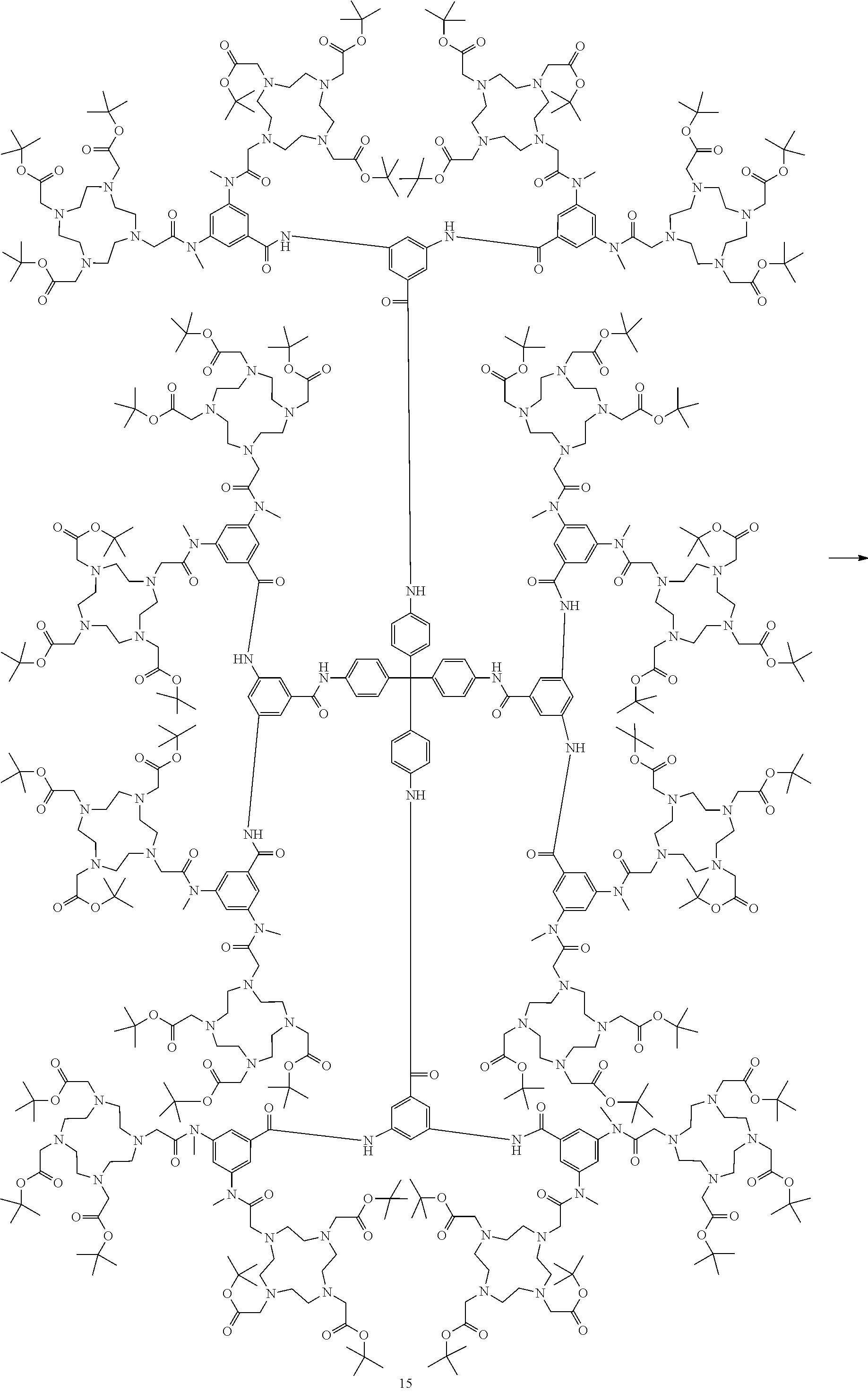

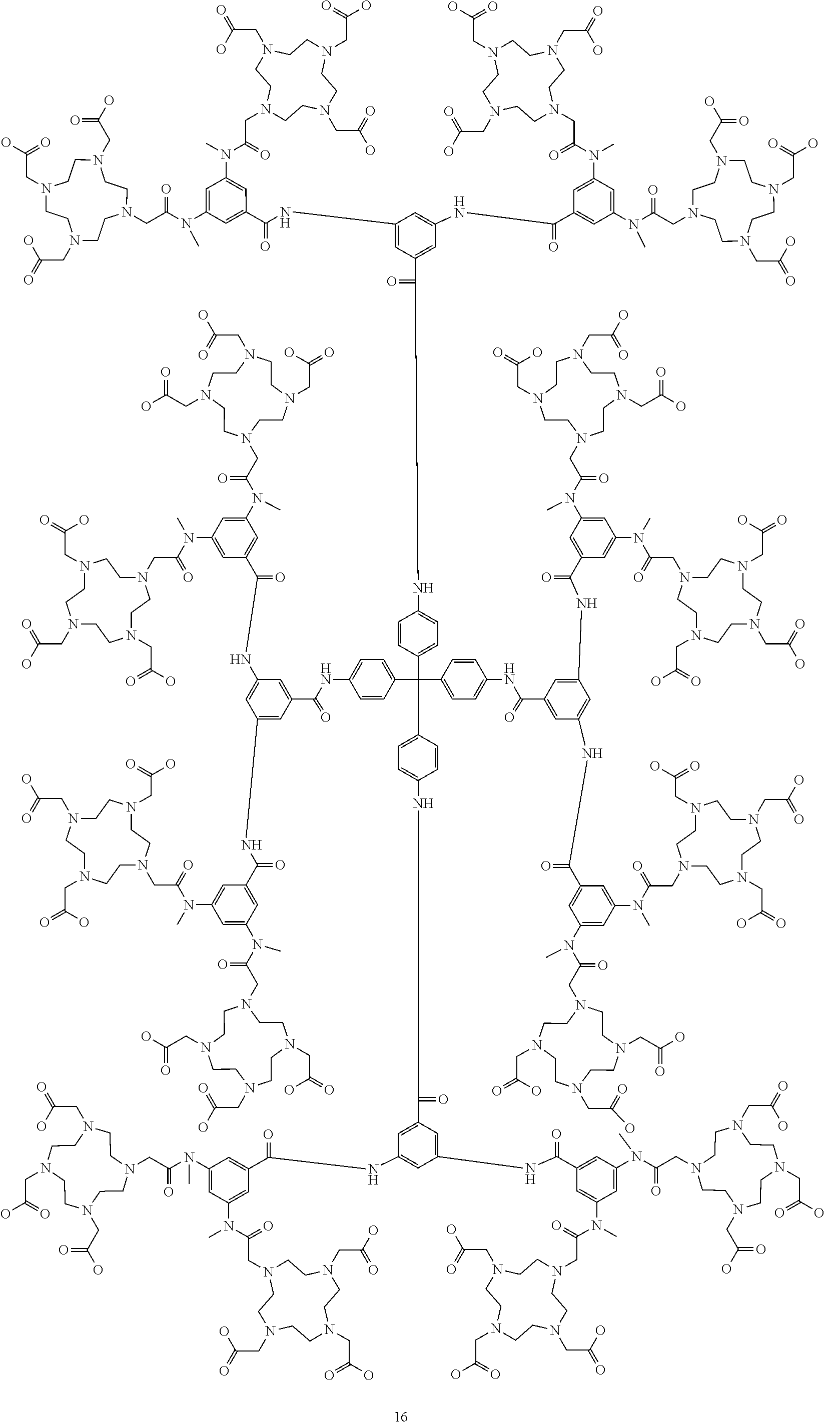

Crude compound 15 (3.1 g) was dissolved in formic acid (50 mL) and refluxed for 15 min under argon atmosphere. The reaction mixture was concentrated to give crude 16. The structure was confirmed by LC-TOF.

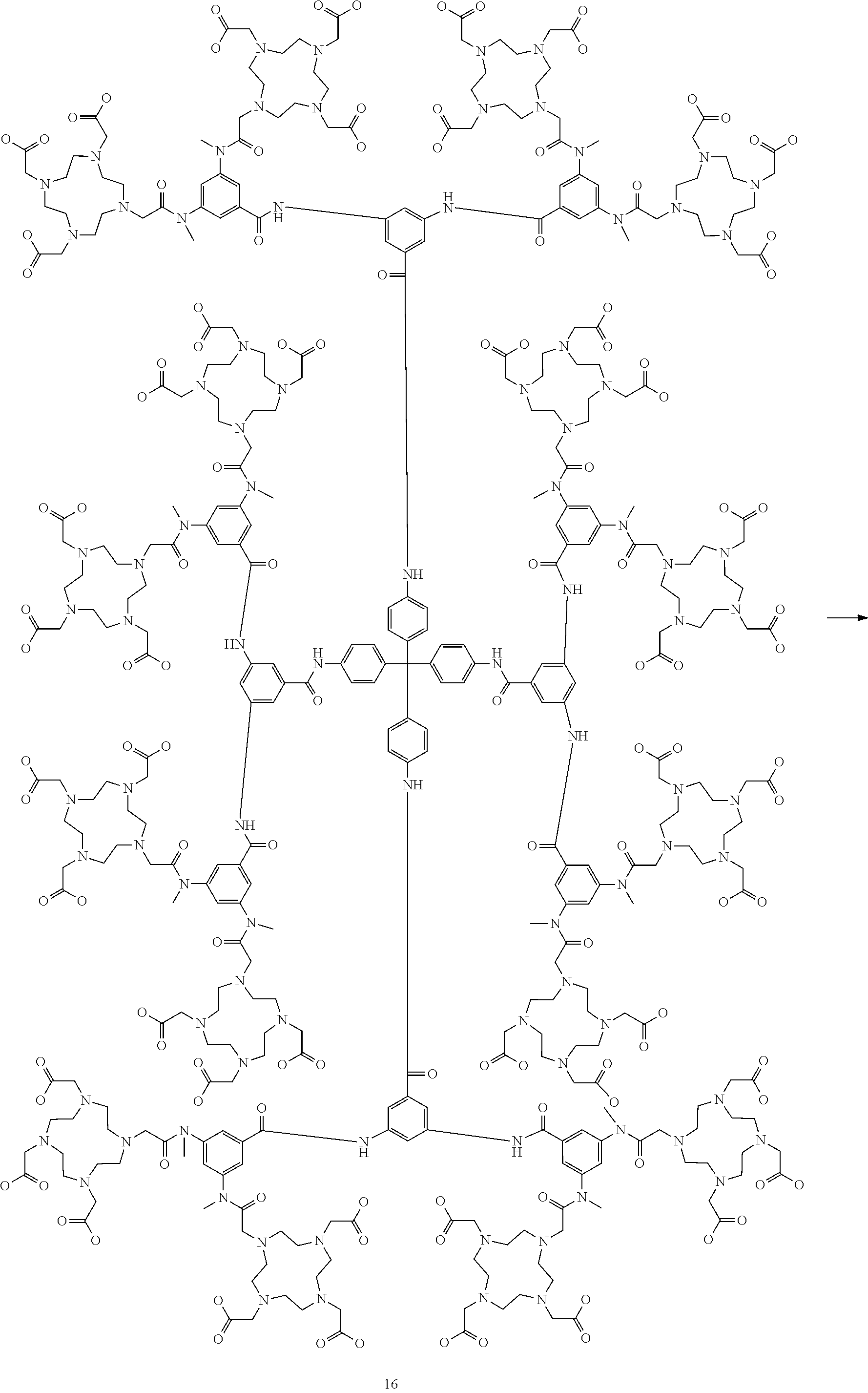

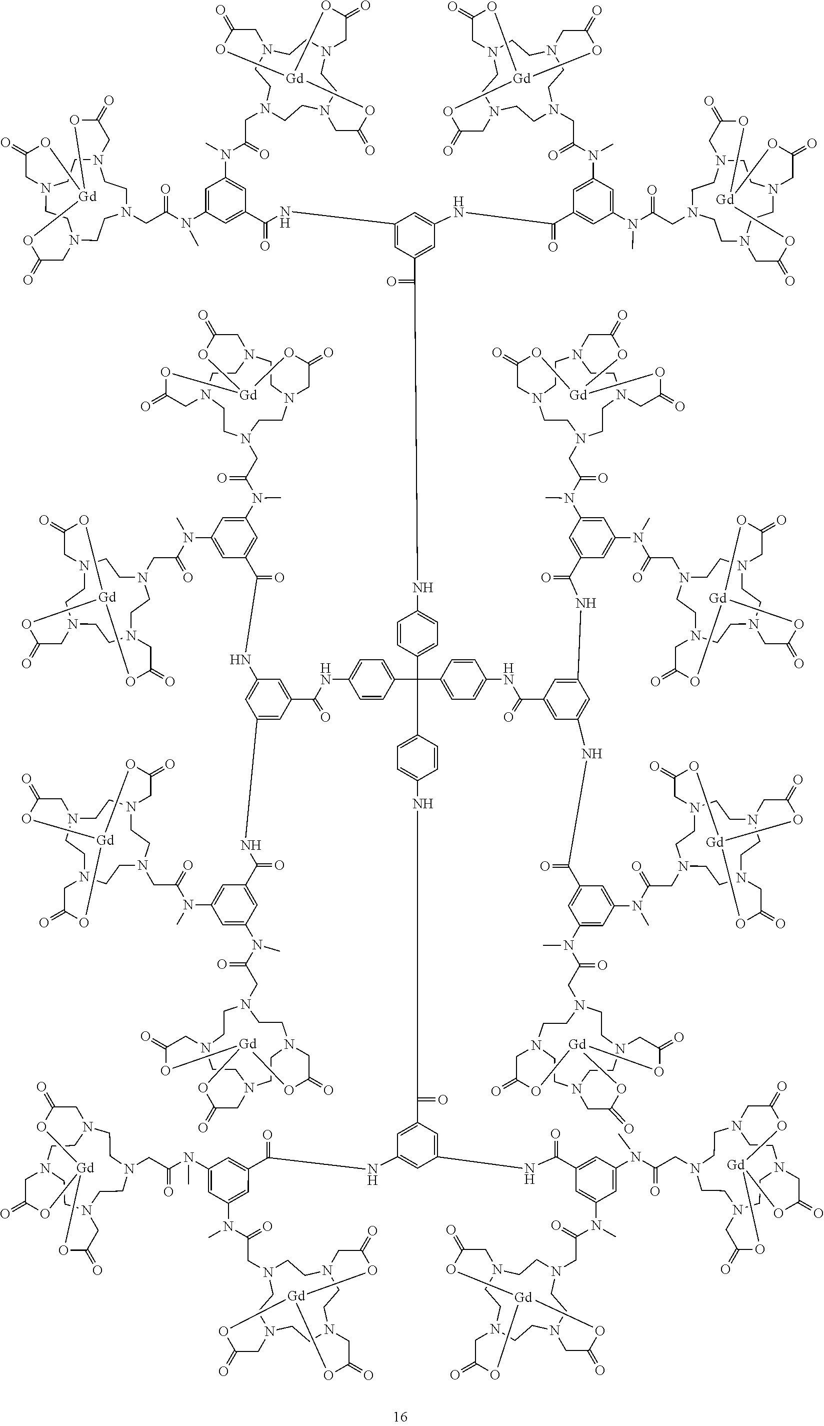

Crude 16 was dissolved in NaOAc buffer (20 mL, 0.5M) and Gd(OAc)3 (3.95 g, 11.81 mmol) was added. pH was adjusted from 4 to 5 by addition of NaOAc (0.5 g). After 3 h the crude reaction mixture was filtered using 10 kD filters (centricon Plus-20, 10 kD filters, Millipore). The retentate was diluted with water (to 18 mL) and filtered again. This procedure was repeated six times and then the retentate was concentrated to give crude 17 (1.4 g). After preparative purification (together with a second 1.4 g batch) 1.1 g (35% over three steps) of pure 17 was obtained. The structure was confirmed by LC-TOF.

### Example 2

### Synthesis of a Compound According to the Present Invention with Formula A-(B-L-R-(L′-X′)₂)₄

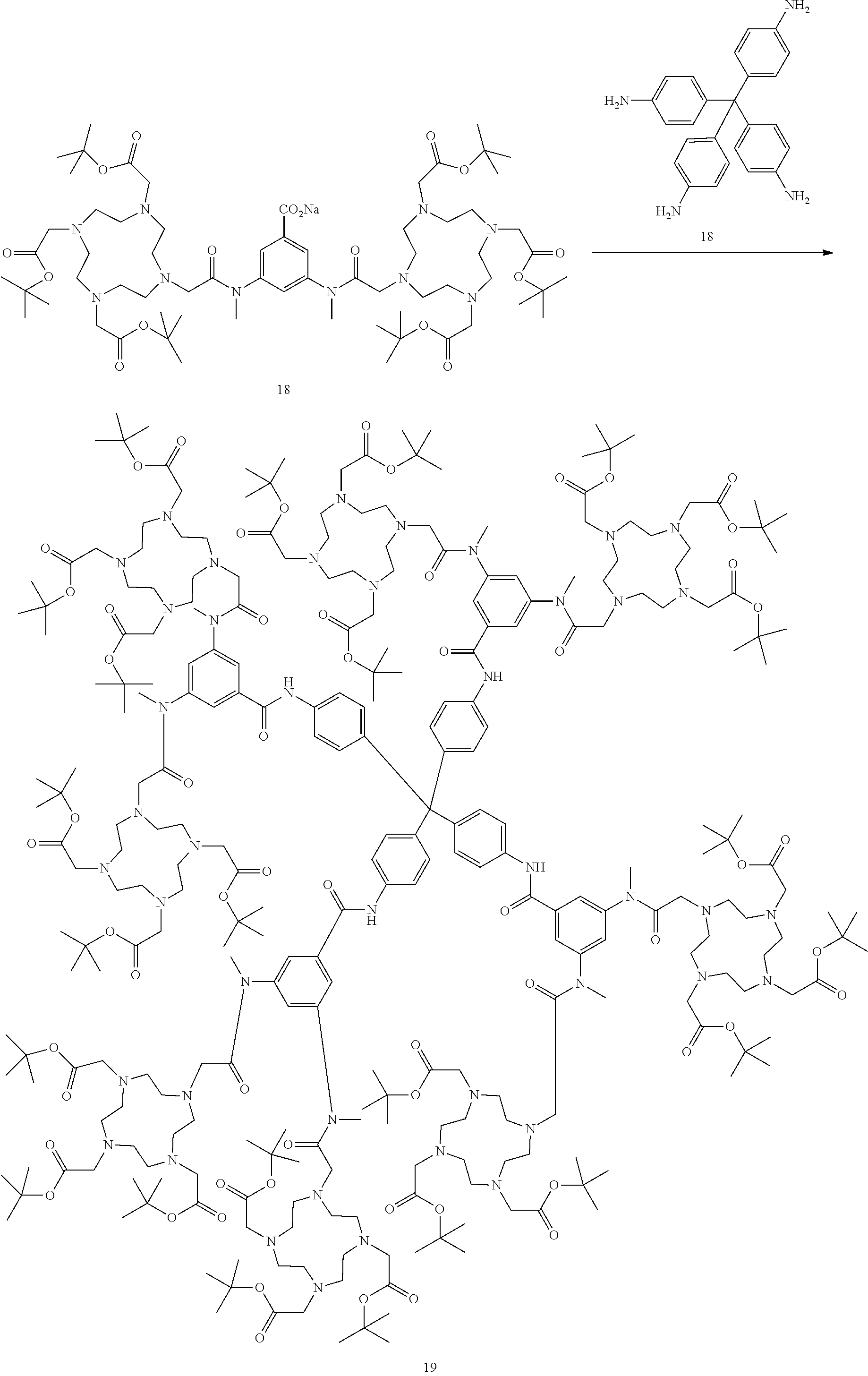

Compound 18 (5 g, 3.88 mmol) was dissolved in dichloromethane (50 mL, dried MS 4 Å) and molecular sieves 4 Å (3 g), HCl (3.9 mL, 4M dioxane, 15.6 mmol) and triphenylphosphine dichloride (3.3 g, 9.90 mmol) were added. The reaction mixture was stirred under nitrogen atmosphere at room temperature for 30 min and then additional triphenylphosphine dichloride (3.3 g, 9.90 mmol) was added. After 30 min compound 14 (246 mg, 0.65 mmol) was added followed by triethylamine (2.7 mL, 19.4 mmol). After 16 h THF (20 mL, dried neutral Al₂O₃) was added and after 23 h additional triethylamine (1.35 mL, 9.7 mmol) was added. After 40 h of reaction time the mixture was filtered and concentrated to give crude compound 19. The structure was confirmed by LC-MS.

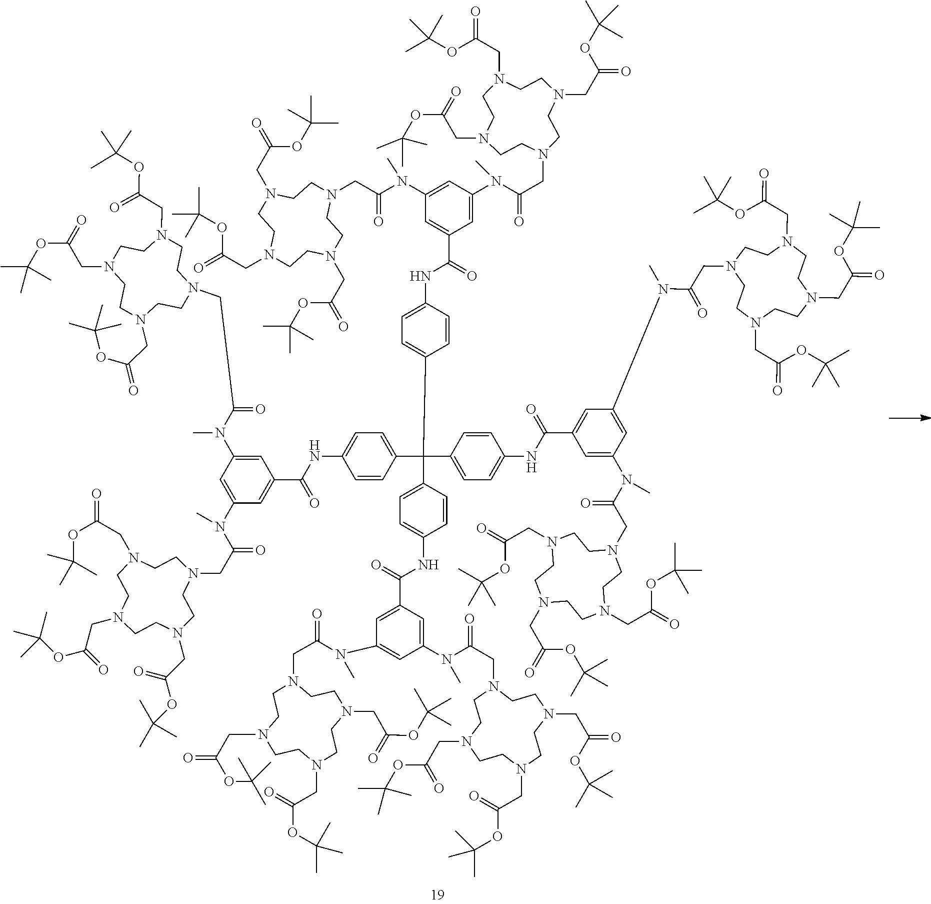

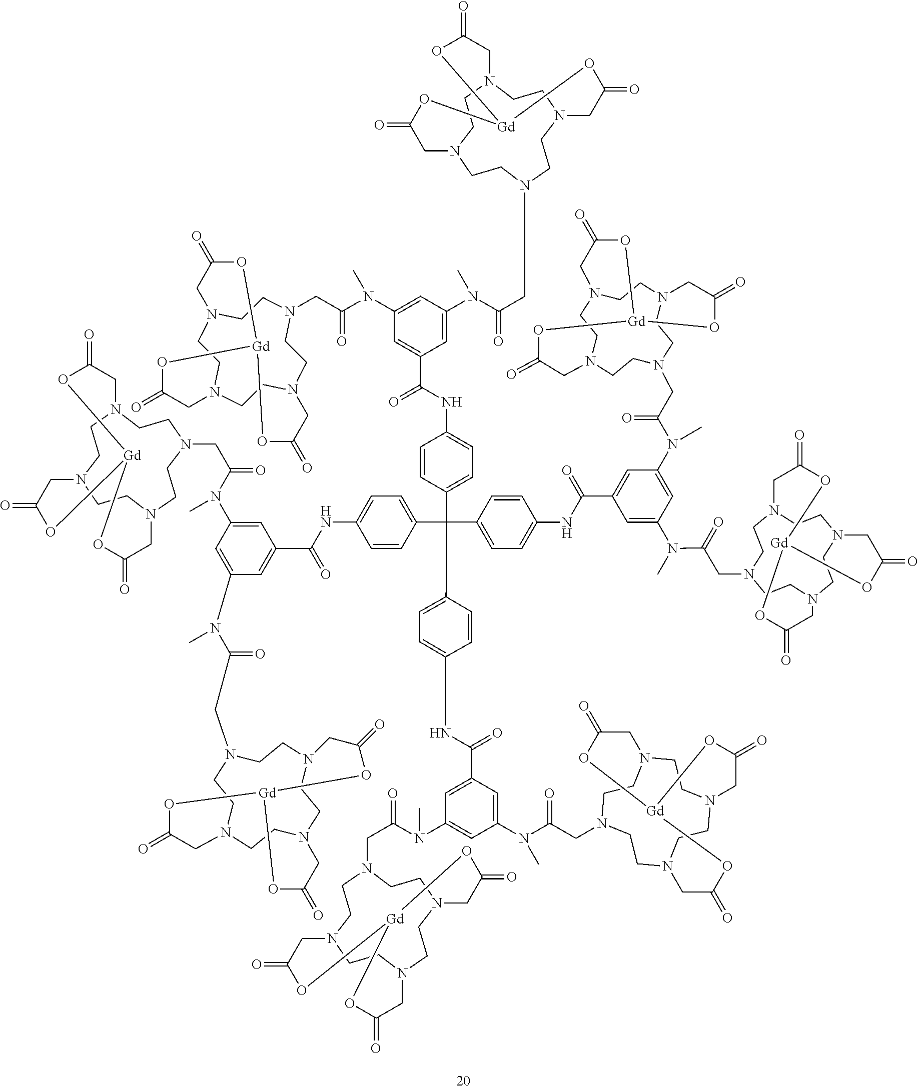

To crude compound 19 was added formic acid (50 mL) and the resulting slurry was heated to 80° C. under nitrogen atmosphere and then cooled to room temperature in 30 min cycles. The heating/room temperature cycling was repeated three times and then the reaction mixture was concentrated. To the crude reaction mixture was added Gd(OAc)₃ (5.3 g, 16 mmol) followed by water (50 mL) and acetonitirile (50 mL). The pH of the formed brown slurry was adjusted from 3 to 5 by addition of NaOAc (6 g, 73 mmol). The acetonitrile was evaporated off and the formed precipitate (Ph₃PO) was filtered off. Then additional Gd(OAc₃) (3 g, 9 mmol) was added. After 1 h the reaction mixture was diluted with water (100 mL) and filtered on YM-3 Millipore filters (Amicon, centiprep 3 kD filters). The obtained retentate was diluted with water (from 55 mL to 100 mL) and filtered again. This procedure was repeated again and then the retentate) was concentrated to give crude compound 20 (4.2 g). Crude 20 was purified on preparative HPLC to give 700 mg (20% over three steps) of pure compound. The structure was confirmed by LC-TOF analysis.

1. Compound of formula (II)
A-(B-L-R-(L-R-(L-R-(L-R-(L′-X′)ᵣ)ᵣ)ᵣ)ᵣ)ₙ (II)
wherein
A denotes a core;
B is the same or different and denotes a moiety that constitutes an obstacle for the rotation of the covalent bond between B and L.
L is the same or different and denotes a rigid linker moiety, wherein at least one L is present and under the proviso that L never links directly to another L;
L′ is present or not and if present is the same or different and denotes a linker moiety;
R is the same or different and denotes a branching moiety that reproduces with an individual multiplicity of r, wherein at least one R is present;
X′ is the same or different and denotes a paramagnetic chelate consisting of a chelator X and a paramagnetic metal ion M; and
r is the same or different and denotes the integer 2, 3 or 4; and
n denotes an integer of 3 to 6.

2. Compound according to claim 1 wherein L is selected from the group consisting of 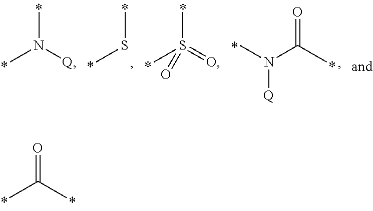
wherein
Q stands for H, C₁-C₈-alkyl, optionally substituted with one or more hydroxyl or amino groups; and
* stands for the possible attachment points to B and R.

3. Compound according to claim 1 wherein A is a cyclic core, a carbon atom or an ethyl group.

4. Compound according to claim 1 wherein A is a saturated or non-saturated, aromatic or aliphatic ring comprising at least 3 carbon atoms and optionally one or more heteroatoms N, S or O, said ring being optionally substituted with one or more of the following substituents: C₁-C₃-alkyl, optionally substituted with hydroxyl or amino groups, amino or hydroxyl groups or halogen, provided that there are n attachment points left for groups B.

5. Compound according to claim 1 wherein B is a moiety whose rotation is hindered by sterical interaction with L.

6. Compound according to claim 1 wherein B comprises a benzene residue.

7. Compound according to claim 1 wherein the B moieties are interconnected by covalent bonds.

8. Compound according to claim 1 wherein R is a moiety whose rotation is hindered by sterical interaction with L and or L′.

9. Compound according to claim 1 wherein R is selected from the group consisting of 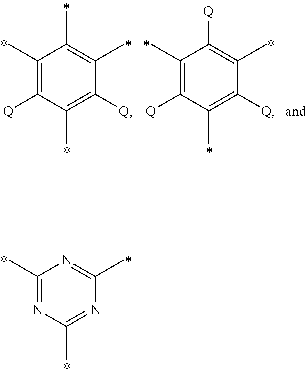
wherein Q is H or CH₃.

10. Compound according to claim 1 wherein X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

11. Compound according to claim 1 wherein M is a paramagnetic ion of a transition metal or a lanthanide metal.

12. Compound according to claim 1 wherein all B are the same and/or all L are the same and/or all R are the same and/or all L′ are the same and/or all X′ are the same.

13. Composition comprising the compound according to claim 1 and at least one physiologically tolerable carrier.

14. Composition according to claim 13 for use as MR imaging agent or MR spectroscopy agent.

15. (canceled)

16. Method of MR imaging and/or MR spectroscopy wherein the composition according to claim 13 is administered to a subject and the subject is subjected to an MR procedure wherein MR signals are detected from the subject or parts of the subject into which the composition distributes and optionally MR images and/or MR spectra are generated from the detected signals.

17. Method of MR imaging and/or MR spectroscopy wherein a subject which had been previously administered with the composition according to claim 13 is subjected to an MR procedure wherein MR signals are detected from the subject or parts of the subject into which the composition distributes and optionally MR images and/or MR spectra are generated from the detected signals.

18. Compounds of formula (I)
A-(B-L-R-(L-R-(L-R-(L-R-(L′-X)ᵣ)ᵣ)ᵣ)ᵣ)ₙ (I)
wherein
A denotes a core;
B is the same or different and denotes a moiety that constitutes an obstacle for the rotation of the covalent bond between B and L.
L is the same or different and denotes a rigid linker moiety, wherein at least one L is present and under the proviso that L never links directly to another L;
L′ is present or not and if present is the same or different and denotes a linker moiety;
R is the same or different and denotes a branching moiety that reproduces with an individual multiplicity of r, wherein at least one R is present;
X is the same or different and denotes a chelator;
r r is the same or different and denotes the integer 2, 3 or 4; and
n denotes an integer of 3 to 6.
